(19) United States
(12) Patent Application Publication
(10) Pub. No.: US 2024/0209407 A1
(43) Pub. Date: Jun. 27, 2024

(54) COMPOSITIONS AND METHODS FOR PHOSPHORAMIDITE-FREE ENZYMATIC SYNTHESIS OF NUCLEIC ACIDS

(71) Applicant: CAMENA BIOSCIENCE LIMITED, Essex (GB)

(72) Inventors: Derek STEMPLE, Alton, NH (US); Sylwia MANKOWSKA, Essex (GB); Neil BELL, Essex (GB)

(73) Assignee: CAMENA BIOSCIENCE LIMITED, Essex (GB)

(21) Appl. No.: 18/556,773

(22) PCT Filed: Apr. 26, 2022
(86) PCT No.: PCT/US2022/026333

§ 371 Date: Oct. 23, 2023

Related U.S. Application Data
(60) Provisional application No. 63/179,828, filed on Apr. 26, 2021.

(51) Int. Cl. C12P 19/34; C12N 9/00; C12N 9/22; C12N 15/11

(52) U.S. Cl. CPC C12P 19/34; C12N 9/22; C12N 9/93; C12N 15/11; C12Y 605/01001; C12N 2310/16; C12N 2310/531

(57) ABSTRACT

Described herein are compositions of matter and methods to synthesize any nucleic acid (NA) sequence using completely natural nucleic acid sources without the need for large-scale phosphoramidite-mediated chemical synthesis.

### CROSS-REFERENCE TO RELATED APPLICATIONS

This application claims priority to, and the benefit of, U.S. Provisional Application No. 63/179,828, filed Apr. 26, 2021, the contents of which are incorporated herein by reference in their entireties for all purposes.

### SEQUENCE LISTING

The instant application contains a Sequence Listing which has been submitted in ASCII format via EFS-Web and is hereby incorporated by reference in its entirety. Said ASCII copy, created on Apr. 26, 2022, is named “DNWR-009_001WO_SeqList.txt” and is about 17,420 bytes in size.

### BACKGROUND

There is a need in the art for the synthesis of nucleic acids that significantly reduces the error rate and organic waste generation associated with phosphoramidite-mediated chemical synthesis. Additionally, there is a need in the art for synthesis of nucleic acids at a significantly reduced cost. Described herein are compositions of matter and methods to synthesize any nucleic acid (NA) sequence using completely natural nucleic acid sources without the need for large-scale phosphoramidite-mediated chemical synthesis.

### SUMMARY

The present disclosure provides one or more Addamers where each carries a payload of at least three base pairs (bp). The full set of 3-mer donor or acceptor Addamer designs includes, as payload, all 64 3-mer bp possibilities. Other embodiments have similar hairpin designs with increased length of the payload. With a payload of three, there are 64 possible Addamers. With a payload of 4 bp there are 256 possible Addamers in a given library. Likewise, for 5-mer payloads there are 1,024 elements and for 6-mer payloads, 4,096 elements. As Addamer are double stranded, through placement of various design features it is possible to use fewer than the all-possible N-mer maximum for a given library and still achieve complete sequence coverage.

Addamers can comprise hairpin turns of several bases and a stretch of short sequence, which can be used to guide the cleavage of the payload from the hairpin region with endonucleases. In addition, to facilitate attachment to solid supports, the hairpin regions can comprise any of several possible specific structural sequences, such as: aptamer sequences for affinity purification or attachment; enzymatic sequences (DNAzymes) for controlled autocleavage; nested endonuclease recognition sites, for attachment by sticky-end ligation to solid supports; or unpaired single-stranded regions for attachment by hybridization to a solid-support bound anchor sequence.

Addamers can comprise a variety of offset cutting Type II S restriction endonuclease sites to enable a variety of overhang lengths or to allow a whole given construct to be cut to a specific shorter length. Differential left versus right side endonuclease site allow for generation of specific ‘donor’ versus ‘acceptor’ Addamer intermediates as longer NA sequences are generated. As would be appreciated by the skilled artisan, hundreds of Type II S restriction endonucleases (IISREs) have been identified. They are characterized by the fact that the double stranded DNA recognition site is separated from the site of cleavage. As methods for oligonucleotide synthesis from Addamer rely on ligation, 5′ overhangs are preferred and there are a variety of IISREs that yield 5′ overhangs of 1, 2, 3, 4 and 5 bases as well as blunt ends.

In one embodiment an array of unique Acceptor and Donor starting payloads are generated by sequential attachment of 3-mer Addamer. Initially, in the loading phase, distinct solid surfaces, which have been pre-loaded with double-stranded multiple cloning site (MCS) bearing ‘attachment studs’ are digested with an appropriate restriction endonuclease (RE), such as BamHI. Similarly, first 3-mer Addamers are RE digested and ligated to the ‘attachment studs.’ After exonuclease treatment and rinsing attached Addamer constructs are digested with appropriate blunt cutting IISREs, yielding a blunt-ended, either an attached Addamer with an exposed 3-mer sequence at its end (Acceptor) or a free blunt-ended Addamer with an exposed 3-mer at its end (Donor). Donor Addamer containing solutions are transferred to desired Acceptor Addamer containing wells and the two Addamers are blunt ligated together using human DNA Ligase III (hLig3), which has a high efficiency of >60% for blunt ligation. After ligation exonuclease is applied to the well to remove unreacted sites and the well is rinsed. At this point the well contains solid-support bound Addamer carrying hexamer payloads (see FIG. 8). Hexamer payloads can then be combined through application of appropriate IISREs to generate Acceptor and Donor versions. Donor solutions are then applied to Acceptor wells and ligated via sticky ends to generate an elongated payload, which is then exonuclease treated and rinsed leaving the desired intermediate payload. This reaction cycle is repeated, and payload lengths increased until a payload of desired NA sequence is generated (see FIG. 8).

The present disclosure provides Addamer libraries comprising 64 possible 3-mer, 256 possible 4-mer, 1,024 possible 5-mer or 4,096 possible 6-mer payloads with the following features: a) d double-stranded double-hairpin overall structure; and b) unique left-side and right-side Type II S restriction endonuclease (RE) sites that allow generation of 0, 1, 2, 3, 4, or 5 bp overhangs

In some aspects, hairpin regions can comprise structural features such as aptamers, which may be used to affix the Addamer to solid supports via molecular affinity for aptamer ligands In some aspects, hairpin regions can comprise restriction endonuclease sites for restriction endonuclease cleavage to allow Addamer ligation to nucleic acids previously affixed to solid supports. In some aspects, hairpin regions can comprise lambda phage cos sites that can be cleaved by lambda terminase to allow Addamer ligation to nucleic acids previously affixed to solid supports In some aspects, hairpin regions can comprise single-stranded regions that may be hybridized to nucleic acids previously affixed to solid supports

The present disclosure provides nucleic acid sequences derived from the combination of Addamers after restriction endonuclease cleavage and subsequent ligation.

The present disclosure provides double stranded DNA anchor sequences with 5′ end modifications to attach to solid supports, containing multiple cloning sites, a hairpin structure and a modified, exonuclease-resistant 3′ end.

The present disclosure provides single stranded DNA anchor sequences with 5′ end modifications to attach to solid supports containing a sequence complementary to appropriate Addamer sequences and a modified, exonuclease-resistant 3′ end

The present disclosure provides a method for the generation of Addamer libraries comprising: a) designing of the library elements including the sequence and placement of hairpins, Type II S restriction endonuclease sites, payloads, other restriction endonuclease sites and target sites for insert excision; b) cloning each of the different Addamer elements of the library into a high copy number plasmid, either as a single copy or a multiple copy insert; c) purifying each plasmid or bacteriophage DNA; d) excising the insert using at least one of the following: i) Nickase; ii) Cas9 Nickase with appropriate guide RNAs; and iii) Trans or Cis acting DNAzymes; e) Ligating the inserts to generate Addamer structures; and f) optionally, performing treatment to purify the Addamers.

The present disclosure provides a method for the generation of Addamer libraries comprising: a) designing the library elements including the sequence and placement of hairpins, Type II S restriction endonuclease sites, payloads, other restriction endonuclease sites and target sites for insert excision; b) performing phosphoramidite synthesis of separate top and bottom strands of the specific Addamers; c) optional treatment of pre-Addamer duplexes with MutS or similar error-correcting enzyme; d) contacting the top and bottom strands of the specific Addamers with a ligase enzyme to generate Addamer structure; and e) treating the products of step (d) with exonuclease to purify functional Addamers.

The present disclosure provides a method for the attachment of one or more Addamers to solid supports comprising: a) loading double stranded anchor sequences containing 5′ modification onto a solid surface; b) performing a bacteriophage lambda terminase digestion of anchor sequences; c) performing a bacteriophage lambda terminase digestion of the one or more Addamers; d) incubating the digested one or more Addamers with the digested anchor sequences; and e) ligating the digested one or more Addamers and the digested anchor sequences.

The present disclosure provides a method for the attachment of one or more Addamers to solid supports comprising: a) loading double stranded anchor sequences containing 5′ modification onto a solid surface; b) performing a restriction endonuclease digestion of anchor sequences; c) preforming restriction endonuclease digestion of the one or more Addamers; d) incubating the digested one or more Addamers and the digested anchor sequences; ligating the digested one or more Addamers and the digested anchor sequences.

The present disclosure provides a method for attachment of one or more Addamers comprising an aptamer sequence to solid supports comprising: a) attaching at least one chemical ligand to solid support, wherein the at least one chemical ligand binds to the aptamer sequence; and b) incubating the one or more Addamers and the solid support, thereby attaching the one or more Addamers to the solid support.

The present disclosure provides a method of nucleic synthesis comprising: a) attaching one or more Addamers to at least one solid support; b) performing independent restriction enzyme digestions of Acceptor and Donor Addamers by appropriate IISREs; c) washing the Acceptor Addamer reaction volume; d) incubating the Donor Addamer solution with the Acceptor Addamer reaction volume; e) Ligating the digested Donor and Acceptor Addamers; f) performing an exonuclease digestion; and g) optionally washing the products of step (f). The resulting product can be used as either Acceptor or Donor Addamers in subsequent steps as a. through g. are repeated until the desired final product is generated.

The present disclosure provides a double-stranded Addamer, wherein the Addamer comprises a) a first Type II S restriction endonuclease (IISRE) sequence; b) an N-mer sequence; c) an at least second IISRE sequence; and wherein at least one end of the Addamer comprises a hairpin structure. In some aspects, an Addamer can comprise a hairpin structure at both ends of the Addamer. In some aspects, an Addamer can comprise a) a first IISRE sequence; b) a second IISRE sequence; c) an N-mer sequence; and d) an at least third IISRE sequence. In some aspects, an Addamer can comprise a) a first IISRE sequence; b) a second IISRE sequence; c) an N-mer sequence; d) a third IISRE sequence; and e) an at least fourth IISRE sequence. In some aspects, an Addamer can further comprise a multiple cloning site (MCS) sequence, wherein the MCS sequence comprises one or more restriction endonuclease sequences.

In some aspects, a IISRE sequence can be selected from a MlyI sequence, a NgoAVII sequence, SspD5I sequence, an AlwI sequence, a BccI sequence, a BcefI sequence, a PleI sequence, a BceAI sequence, a BceSIV sequence, a BscAI sequence, a BspD6I sequence, a FauI sequence, an EarI sequence, a BspQI sequence, a BfuAI sequence, a PaqCI sequence, an Esp3I sequence, a BbsI sequence, a BbvI sequence, a BtgZI sequence, a FokI sequence, a BsmFI sequence, a BsaI sequence, a BcoDI sequence and a HgaI sequence.

In some aspects, a hairpin structure can comprise an aptamer sequence. In some aspects, an aptamer sequence can be selected from a pL1 aptamer sequence, a Thrombin 29-mer aptamer sequence, an S2.2 aptamer sequence, an ART1172 aptamer sequence, an R12.45 aptamer sequence, a Rb008 aptamer sequence and a 38NT SELEX aptamer sequence.

The present disclosure provides a composition comprising the Addamer of the presented disclosure immobilized to a solid support. In some aspects, a solid support can be a bead. In some aspects, a bead can comprise polyacrylamide, polystyrene, agarose or any combination thereof. In some aspects, a solid support can be the surface of a well or chamber. In some aspects, a well or chamber can be part of a multi-well plate.

In some aspects wherein an Addamer is immobilized to a solid support, the Addamer comprises a hairpin structure comprising at least one aptamer sequence, the solid surface comprises at least one ligand that binds to the aptamer sequence, and the Addamer is immobilized to the solid surface via binding of the at least one aptamer sequence to the at least one ligand.

In some aspects wherein an Addamer is immobilized to a solid support the Addamer comprises at least one 5′ overhang or 3′ overhang, the solid surface comprises at least one single-stranded or partially double-stranded nucleic acid molecule with a single-stranded portion that is complementary to the at least one 5′ overhang or 3′ overhang, and the Addamer is immobilized to the solid surface by hybridizing the at least one 5′ overhang or 3′ overhang to the at least one single-stranded or partially double-stranded nucleic acid on the solid surface.

In some aspects wherein an Addamer is immobilized to a solid support, the Addamer comprises at least one 5′ overhang or 3′ overhang, the solid surface comprises at least one single-stranded or partially double-stranded nucleic acid molecule with a single-stranded portion that is complementary to the at least one 5′ overhang or 3′ overhang, and the Addamer is immobilized to the solid surface by hybridizing the at least one 5′ overhang or 3′ overhang to the at least one single-stranded or partially double-stranded nucleic acid on the solid surface and ligating the Addamer and the at least one single-stranded or partially double-stranded nucleic acid.

The present disclosure provides a method of producing the Addamer of the present disclosure, the method comprising: a) chemically synthesizing a first single-stranded nucleic acid molecule and a second single-stranded nucleic acid molecule, wherein the sequences of the first single-stranded nucleic acid molecule and second single-stranded nucleic acid molecule comprise portions of the Addamer that is to be produced, wherein the first single stranded nucleic acid molecule comprises a first region that is complementary to a second region on the second single-stranded nucleic acid molecule and the second region that is self-complementary, and the second single-stranded nucleic acid molecule comprises a first region that is self-complementary and a second region that is complementary to a first region on the first single-stranded nucleic acid molecule; b) hybridizing the first single-stranded nucleic acid and the second single-stranded nucleic acid to produce a double-stranded nucleic acid molecule; c) contacting the double-stranded nucleic acid molecule with a ligase enzyme to form a double-stranded Addamer structure capped at both ends by hairpins. In some aspects the preceding method can further comprise treating the products of step (c) with an exonuclease, thereby purifying properly ligated Addamers. In some aspects, the preceding method can further comprise, after step (b) and before step (c), contacting the partially double-stranded nucleic acid molecule with a MutS enzyme.

The present disclosure provides method of producing the Addamer of the present disclosure, the method comprising: a) cloning the Addamer sequence into a phagemid such that the Addamer sequence is flanked on both sides by one or more DNAzymes that can be selectively activated; b) converting the phagemid into packaged bacteriophage using a helper phage, wherein the packaged bacteriophage produces single-stranded DNA comprising the Addamer sequence flanked on both sides by one or more DNAzymes; c) purifying the single-stranded DNA produced by the packaged bacteriophage; d) allowing the purified single-stranded DNA to fold to produce the one or more DNAzyme structure and a majority portion of the double-stranded Addamer sequence; e) activating the one or more DNAzymes, thereby excising the Addamer sequence from the single-stranded DNA produced by the packaged bacteriophage; f) contacting the excised Addamer with a ligase enzyme. The preceding method can further comprise treating the products of step (f) with exonuclease, thereby purifying properly ligated Addamers.

The present disclosure provides method of producing the Addamer of the present disclosure, the method comprising: a) cloning the Addamer sequence into a plasmid; b) propagating the plasmid in a suitable host organism; c) purifying the plasmid from the host organism; d) treating the purified plasmid with one or more of a nickase enzymes and a restriction endonuclease enzyme, or simply one or more nickase enzymes to excise the Addamer sequences from the plasmid; and e) contacting the excised Addamer sequences with ligase to produce double-stranded Addamer structures capped at both ends by a hairpin structure.

The present disclosure provides a method of synthesizing a nucleic acid molecule comprising a target nucleic acid sequence, the method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of any one of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and a 3′ overhang, a 5′ overhang or a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, a 3′ overhang, a 5′ overhang or a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) until the nucleic acid molecule comprising the target nucleic acid sequence has been synthesized.

In some aspects, a ligase enzyme can be human DNA ligase III (hLig3). In some aspects, a ligase enzyme can be T4 DNA ligase.

In some aspects, a target nucleic acid sequence can be at least about 100, or at least about 500, or at least about 1000, or at least about 2000, or at least about 3000, or at least about 4000, at least about 5000 nucleotides in length.

In some aspects, a nucleic acid molecule comprising the target nucleic acid sequence synthesized by the methods of the present disclosure can have a purity of at least 80% or at least 90%.

### BRIEF DESCRIPTION OF THE DRAWINGS

The above and further features will be more clearly appreciated from the following detailed description when taken in conjunction with the accompanying drawings.

FIG. 1 is an exemplary schematic of an Addamer, which is a double-stranded nucleic acid molecule comprising a hairpin at both ends.

FIG. 2 shows exemplary schematics of various Addamer designs of the present disclosure. Show are several examples of Addamer design each Addamer type possesses a payload with flanking IISRE sites, a means of attachment to a solid support and double hairpins to promote exonuclease resistance. Shown in the key are some possible IISRE and RE sites as well as the thrombin aptamer hairpin.

FIG. 3 shows the sequences of 6 non-limiting examples of Addamers of the present disclosure. The Addamer designs comprise simple hairpins, a multiple cloning site and paired IISRE binding sites. In this case each has one blunt cutting IISRE, MlyI, which will leave end of the 3-mer payload, NNN, exposed for blunt ligation. On the left side of each design is a 5′ 4-base overhang or 3-base overhang IISRE site for downstream payload elongation reactions. The nucleotide sequences of ten or more nucleotides presented in FIG. 3 correspond to those put forth in SEQ ID NOs: 1-12.

FIG. 4 show exemplary nested Type II S restriction endonuclease (IISRE) sequences (hereafter “IISRE sequence”) for use in the Addamers of the present disclosure. The nucleotide sequences of ten or more nucleotides presented in FIG. 4 correspond to those put forth in SEQ ID NOs: 13-28.

FIG. 5 is an exemplary schematic of a method of producing an Addamer of the present disclosure. In the method, two distinct oligonucleotides are synthesized and hybridized together. Treatment with MutS, which binds mismatched bases and exposes DNA to exonuclease digestion, is combined with ligation, followed by T7 exonuclease digestion. This process leaves substantially pure Addamer. These Addamer can be evaluated in oligonucleotide synthesis reactions and then serve as templates for clonal production of Addamer. The nucleotide sequences of ten or more nucleotides presented in FIG. 5 correspond to those put forth in SEQ ID NOs: 29-32.

FIG. 6 is a schematic of a phagemid for use in the production of an Addamer of the present disclosure. This phagemid construct comprises the information to generate bacteriophages from double-stranded DNA as well as the DNAzyme for efficient excision of pure Addamers of clonal origin. From a chemically synthesized Addamer, of any payload length, an insert is generated using the For and Rev primers and ligated into the phagemid vector.

FIG. 7A is an exemplary schematic of a method of producing an Addamer of the present disclosure using a phagemid and DNAzymes. Depicted is a schematic of a specific Addamer 3-mer payload GCC in an Addamer design that includes nested IISREs (blunt cutting site nested with 4-base overhang site and blunt site nested with 4-base site) and paired MCS (MSC left and MCS right). This design also includes forward and reverse amplification primer sites (For and Rev) as well as DNAzyme scar sequences. Also depicted are flanking DNAzyme pairs. Single stranded DNA produced by bacteriophage propagation in E. coli is purified and allowed to fold to produce DNAzyme structures and the majority portion of the double-stranded Addamer sequence. After activation by treatment with Zn+, ligation and T7 exonuclease treatment pure clonal Addamer is produced.

FIG. 7B is an exemplary schematic of a method of producing an Addamer of the present disclosure using bimolecular, trans cleavage excision of an Addamer sequence.

FIG. 8 is an exemplary schematic of a nucleic acid synthesis method of the present disclosure that comprises the use of Addamers of the present disclosure. Initially the 1^{st} and 2^{nd} Addamers are attached using DNA ligation to MCS bearing attachments studs, which have already been loaded onto a solid support. Donor and Acceptor constructs are generated in separate volumes. Donor and Acceptor constructs are treated with distinct IISREs to generate ligatable ends. In this diagram, the Acceptor is generated by digestion with the R1 IISRE, the released end and enzyme are discarded by rinsing. The Donor construct is generated by digestion with the purple L2 enzyme. The Donor construct solution (carrying the L2 enzyme) is transferred to the Acceptor well and ligated using T4 DNA Ligase, which has a high efficiency of >80% for 2, 3 or 4-base sticky end ligation. The well is treated with exonuclease and rinsed. The resulting attached Addamer construct is then ready for subsequent cycles of elongation.

FIG. 9A, FIG. 9B, FIG. 9C, FIG. 9D, FIG. 9E, FIG. 9F, FIG. 9G and FIG. 9H are exemplary schematics of a nucleic acid synthesis method of the present disclosure that comprises the use of Addamers of the present disclosure to synthesize a 27 nucleotide long target nucleic acid molecule. The nucleotide sequence of ten or more nucleotides presented in FIG. 9A corresponds to that put forth in SEQ ID NO: 33. The nucleotide sequences of ten or more nucleotides presented in FIG. 9E correspond to those put forth in SEQ ID NOs: 34-35. The nucleotide sequences of ten or more nucleotides presented in FIG. 9G correspond to those put forth in SEQ ID NOs: 36-47. The nucleotide sequences of ten or more nucleotides presented in FIG. 9H correspond to those put forth in SEQ ID NOs: 48-58.

FIG. 10 is a schematic of a set of restriction enzyme digestions reactions and ligation reactions performed using the Addamers of the present disclosure to synthesize a target nucleic acid. The nucleotide sequences of ten or more nucleotides presented in FIG. 10 correspond to those put forth in SEQ ID NOs: 59-67.

FIG. 11 show the results of gel electrophoresis analysis of the enzyme digestion reactions and ligation reactions performed using the Addamers of the present disclosure, as outlined in FIG. 10.

### DETAILED DESCRIPTION

The present disclosure is directed to compositions and methods for the synthesis of nucleic acid molecules comprising specific target nucleic acid sequences. The compositions can comprise Addamers, which are described in more detail herein. The methods can comprise the use of these Addamers in successive restriction enzyme cleavage and ligation reactions to produce nucleic acid molecules comprising target nucleic acid sequences. These methods are described in more detail herein.

Importantly, unlike existing nucleic acid synthesis methods, the compositions and methods of the present disclosure do not require large-scale phosphoramidite synthesis. Consequently, the compositions and methods of the present disclosure are less expensive and faster than existing nucleic acid synthesis methods. Moreover, the compositions and methods of the present disclosure produce less toxic, organic waste, and are therefore more environmentally conscious than existing nucleic acid synthesis methods.

### Addamers

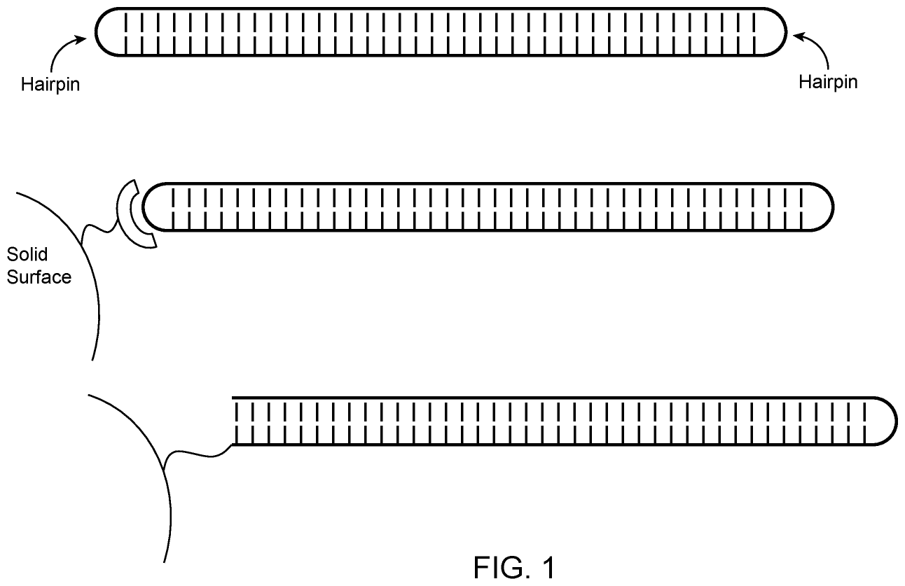

The present disclosure provides a composition comprising at least one Addamer. As used herein, the term Addamer is used to describe a double-stranded nucleic acid molecule comprising a hairpin structure at both ends. In some aspects wherein the Addamer is immobilized to a solid surface, an Addamer may comprise a single hairpin located at the end of the molecule that is not attached to the solid surface. An exemplary schematic of an Addamer and two Addamers immobilized to a solid surface are shown in FIG. 1. An Addamer can comprise one or more features described herein.

In some aspects, an Addamer can comprise, consists essentially of, or consist of DNA.

In some aspects, an Addamer can comprise one or more multiple cloning site (MCS) sequences. In some aspects, an MCS sequence can comprise one or more restriction endonuclease (RE) sequences that can be cleaved with the corresponding restriction endonuclease to generate a 3′ overhang, a 5′ overhang, or a blunt end.

As would be appreciated by the skilled artisan, a “blunt end” is used to describe the end of a DNA fragment in which there are no unpaired nucleotides.

As would be appreciated by the skilled artisan, the term 5′ overhang is used to refer to a single-stranded portion of a partially double-stranded nucleic acid molecule that is located at the 5′ terminus of one of the strands.

As would be appreciated by the skilled artisan, the term 3′ overhang is used to refer to a single-stranded portion of a partially double-stranded nucleic acid molecule that is located at the 3′ terminus of one of the strands.

In some aspects, an Addamer can comprise at least one offset cutting Type II S restriction endonuclease (IISRE) sequences (hereafter “IISRE sequence”) that can be cleaved with a corresponding Type II S restriction endonuclease (hereafter “IISRE”). In some aspects, an Addamer can comprise at least one IISRE sequences. In some aspects, an Addamer can comprise at least three IISRE sequences. In some aspects, an Addamer can comprise at least four IISRE sequences.

In some aspects, the IISRE sequence is a sequence such that cleavage with the corresponding IISRE results in the creation of a “blunt end”.

In some aspects, the IISRE sequence is a sequence such that cleavage with the corresponding IISRE results in the creation of a 5′ overhang that is 1 nucleotide in length. In some aspects, the IISRE sequence is a sequence such that cleavage with the corresponding IISRE results in the creation of a 5′ overhang that is 2 nucleotides in length. In some aspects, the IISRE sequence is a sequence such that cleavage with the corresponding IISRE results in the creation of a 5′ overhang that is 3 nucleotides in length. In some aspects, the IISRE sequence is a sequence such that cleavage with the corresponding IISRE results in the creation of a 5′ overhang that is 4 nucleotides in length. In some aspects, the IISRE sequence is a sequence such that cleavage with the corresponding IISRE results in the creation of a 5′ overhang that is 5 nucleotides in length. In some aspects, the IISRE sequence is a sequence such that cleavage with the corresponding IISRE results in the creation of a 5′ overhang that is about 1 nucleotide to about 5 nucleotides in length.

Non-limiting examples of IISRE sequences, along with their corresponding IISRE and a description of the overhang/blunt end that is created by cleavage of the IISRE sequence and the corresponding IISRE are shown in Table 1. Accordingly, an Addamer can comprise one or more of the IISRE sequences put forth in Table 1.

TABLE 1
Exemplary IISRE sequences and corresponding IISRE

| | | Overhang/Blunt |
|---|---|---|
| IISRE Name | IISRE sequence | end generated |
| MlyI | GAGTC (5/5) | Blunt end |
| NgoAVII | GCCGC (7/7) | Blunt end |
| SspDSI | GGTGA (8/8) | Blunt end |
| AlwI | GGATC (4/5) | 1 nt 5′ overhang |
| BccI | CCATC (4/5) | 1 nt 5′ overhang |
| BcefI | ACGGC (12/13) | 1 nt 5′ overhang |
| PleI | GAGTC (4/5) | 1 nt 5′ overhang |
| BceAI | ACGGC (12/14) | 2 nt 5′ overhang |
| BceSIV | GCTGC (9/11) | 2 nt 5′ overhang |
| BscAI | GCATC (4/6) | 2 nt 5′ overhang |
| BspD6I | GAGTC (4/6) | 2 nt 5′ overhang |
| FauI | CCCGC (4/6) | 2 nt 5′ overhang |
| EarI | CTCTTC (1/4) | 3 nt 5′ overhang |
| BspQI | GCTCTTC (1/4) | 3 nt 5′ overhang |
| BfuAI | ACCTGC (4/8) | 4 nt 5′ overhang |
| PaqCI | CACCTGC (4/8) | 4 nt 5′ overhang |
| Esp3I | CGTCTC (1/5) | 4 nt 5′ overhang |
| BbsI | GAAGAC (2/6) | 4 nt 5′ overhang |
| BbvI | GCAGC (8/12) | 4 nt 5′ overhang |
| BtgZI | GCGATG (10/14) | 4 nt 5′ overhang |
| FokI | GGATG (9/13) | 4 nt 5′ overhang |
| BsmFI | GGGAC (10/14) | 4 nt 5′ overhang |
| BsaI | GGTCTC (1/5) | 4 nt 5′ overhang |
| BcoDI | GTCTC (1/5) | 4 nt 5′ overhang |
| HgaI | GACGC (5/10) | 5 nt 5′ overhang |

In some aspects, a hairpin structure, or hairpin (used interchangeably), located at the end of an Addamer can comprise at least about 1, or at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50 nucleotides.

As described above, Addamers are capped at either end by hairpin structures. The hairpin structures serve several roles. First, the hairpins provide protection against exonuclease digestion for the Addamer. This allows clearance of unreacted intermediates from a given reaction in the methods of the present disclosure, which provides purity, both of Addamers during generation and of products after Addamer elongation. Secondly, the hairpin structures provide means for attachment of Addamers to solid supports. These attachments are generated directly by binding of aptamers, which bind to specific solid-support bound ligands; by ligation through MCS after digestion with conventional REs, such as BamHI; by ligation through lambda phage cos sites, or by hybridization to single stranded solid-support bound anchor NA. Finally, the hairpins described herein allow Addamers to be attached to a solid support (e.g. a bead) without the need for non-natural modifications such as biotin. Thus, the Addamer of the present disclosure can be synthesized using completely natural means, removing the need for small- and or large-scale phosphoramidite synthesis. Accordingly, the Addamers and methods of the present disclosure can allow for faster and less expensive synthesis of nucleic acid molecules and produce less toxic waste.

In some aspects, a hairpin located at the end of an Addamer can comprise a structural sequence that allows for the affinity purification of the Addamer and/or attachment of the Addamer to a solid support (e.g. a bead).

In some aspects, a hairpin located at the end of an Addamer can comprise an enzymatic sequence (e.g. a DNAzyme sequence) that allows for controlled autocleavage.

In some aspects, a hairpin located at the end of an Addamer can comprise one or more restriction enzyme sites. Without wishing to be bound by theory, the one or more restriction enzyme sites in a hairpin can be cleaved with the corresponding restriction enzyme(s) to generate at least one single-stranded overhang, which can subsequently be used to hybridize and/or ligate the cleaved Addamer to a solid support (e.g. a bead) comprising a nucleic acid that is complementary to the at least one single-stranded overhang.

In some aspects, a hairpin located at the end of an Addamer can comprise an aptamer sequence. Without wishing to be bound by theory, an aptamer sequence can be used for affinity purification and/or attachment to a solid support (e.g. a bead). Non-limiting examples of aptamer sequences are shown in Table 2.

TABLE 2
Exemplary Aptamers

| Aptamer | | |
|---|---|---|
| Name | Ligand | Sequence |
| pLI | Anti- | TCGATTGGATTGTGCCGGAAGTGCTGGCTCGA |
| | PvLDH | (SEQ ID NO: 68) |
| Thrombin | Anti- | AGTCCGTGGTAGGGCAGGTTGGGGTGACT |
| 29-mer | thrombin | (SEQ ID NO: 69) |
| S2.2 | Anti- | CAGTTGATCCTTTGGATACCCTG (SEQ ID |
| | Muc1 | NO: 70) |
| ART1172 | Anti-VWF | GGCGTGCAGTGCCTTCGGCCGTGCGGTGCCTC |
| | | CGTCACGCCT (SEQ ID NO: 71) |
| R12.45 | Anti- | ACCGTCTGAGCGATTCGTACTTTATTCGGGAG |
| | Atrazine | GTATCAGCGGG (SEQ ID NO: 72) |
| Rb008 | Anti-ATX | CCTGGACGGAACCAGAATACTTTTGGTCTCCA |
| | | GG (SEQ ID NO: 73) |
| 38NT | Anti- | AAATACCCCCCCTTCGGTGCAAAGCACCGAAG |
| SELEX | HIV RT | GGGGGGTATTT (SEQ ID NO: 74) |

In some aspects, an Addamer can comprise a lambda phage cos site.

In some aspects, an Addamer can comprise an “N-mer sequence” that comprises a fragment of a nucleic acid that is to be synthesized using one of the methods described herein. The terms “N-mer sequence”, “payload” and “N-mer payload” are used herein interchangeably.

In some aspects, an N-mer sequence can be about 3 nucleotides in length. In some aspects, an N-mer sequence is 3 nucleotides in length. An N-mer sequence that is 3 nucleotides in length is herein referred to as a 3-mer.

In some aspects, an N-mer sequence can be about 4 nucleotides in length. In some aspects, an N-mer sequence is 4 nucleotides in length. An N-mer sequence that is 4 nucleotides in length is herein referred to as a 4-mer.

In some aspects, an N-mer sequence can be about 5 nucleotides in length. In some aspects, an N-mer sequence is 5 nucleotides in length. An N-mer sequence that is 5 nucleotides in length is herein referred to as a 5-mer.

In some aspects, an N-mer sequence can be about 6 nucleotides in length. In some aspects, an N-mer sequence is 6 nucleotides in length. An N-mer sequence that is 6 nucleotides in length is herein referred to as a 6-mer.

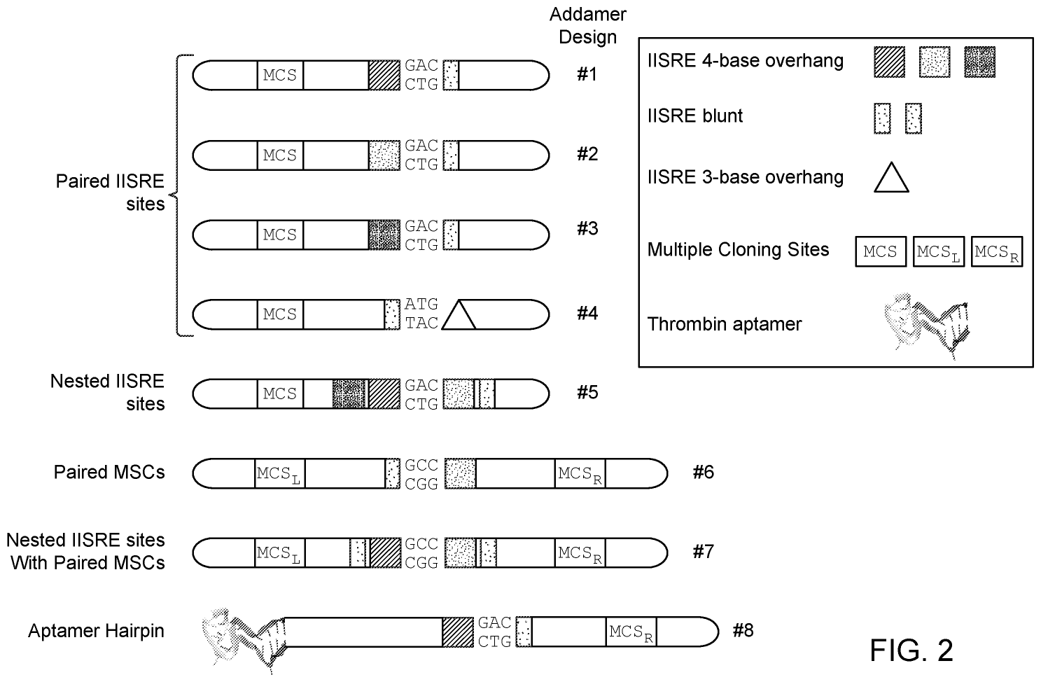

In some aspects, an Addamer can comprise an MCS sequence, a first IISRE sequence, an N-mer sequence, and an at least second IISRE sequence. In some aspects, an Addamer can comprise an MCS sequence, followed by a first IISRE sequence, followed by an N-mer sequence, followed by an at least second IISRE sequence. An exemplary schematic of the preceding Addamer is shown in FIG. 2 as Addamer Designs #1-#4. In the non-limiting examples of Addamer Designs #1-#3 shown in FIG. 2, the first IISRE sequence is an IISRE sequence that when cleaved creates a 4 nucleotide long 5′ overhang, the N-mer sequence is a 3-mer sequence, and the at least second IISRE sequence is an IISRE sequence that when cleaved creates a blunt end. In the non-limiting example of Addamer Design #4 shown in FIG. 2, the first IISRE sequence is an IISRE sequence that when cleaved creates a blunt end, the N-mer sequence is a 3-mer sequence, and the at least second IISRE sequence is an IISRE sequence that when cleaved creates a 3 nucleotide long 5′ overhang.

In some aspects, an Addamer can comprise an MCS sequence, a first IISRE sequence, a second IISRE sequence, an N-mer sequence, a third IISRE sequence and at least fourth IISRE sequence. In some aspects, can comprise an MCS sequence, followed by a first IISRE sequence, followed by a second IISRE sequence, followed by an N-mer sequence, followed by a third IISRE sequence, followed by an at least fourth IISRE sequence. An exemplary schematic of the preceding Addamer is shown in FIG. 2 as Addamer Design #5. In the non-limiting example of Addamer Design #5 shown in FIG. 2, the first IISRE sequence is an IISRE sequence that when cleaved creates a 4 nucleotide long 5′ overhang, the second IISRE sequence is an IISRE sequence that when cleaved creates a 4 nucleotide long 5′ overhang, the N-mer sequence is a 3-mer sequence, the third IISRE sequence is an IISRE sequence that when cleaved creates a 4 nucleotide long 5′ overhang, and the at least fourth IISRE sequence is an IISRE sequence that when cleaved creates a blunt end.

In some aspects, an Addamer can comprise a first MCS sequence, a first IISRE sequence, an N-mer sequence, an at least second IISRE sequence and an at least second MCS sequence. In some aspects, an Addamer can comprise a first MCS sequence, followed by a first IISRE sequence, followed by an N-mer sequence, followed by an at least second IISRE sequence, followed by an at least second MCS sequence. An exemplary schematic of the preceding Addamer is shown in FIG. 2 as Addamer Design #6. In the non-limiting example of Addamer Design #6 shown in FIG. 2, the first IISRE sequence is an IISRE sequence that when cleaved creates a blunt end, the N-mer sequence is a 3-mer sequence, and the at least second IISRE sequence is an IISRE sequence that when cleaved creates a 4 nucleotide long 5-overhang.

In some aspects, an Addamer can comprise a first MCS sequence, a first IISRE sequence, a second IISRE sequence, an N-mer sequence, a third IISRE sequence, an at least fourth IISRE sequence and an at least second MCS sequence. In some aspects, an Addamer can comprise a first MCS sequence, followed by a first IISRE sequence, followed by a second IISRE sequence, followed by an N-mer sequence, followed by a third IISRE sequence, followed by an at least fourth IISRE sequence, followed by an at least second MCS sequence. An exemplary schematic of the preceding Addamer is shown in FIG. 2 as Addamer Design #7. In the non-limiting example of Addamer Design #7 shown in FIG. 2, the first IISRE sequence is an IISRE sequence that when cleaved creates a blunt end, the second IISRE sequence is an IISRE sequence that when cleaved creates a 4 nucleotide long 5′ overhang, the N-mer sequence is a 3-mer sequence, the third IISRE sequence is an IISRE sequence that when cleaved creates a 4 nucleotide long 5′ overhang, and that at least fourth IISRE sequence is an IISRE sequence that when cleaved creates a blunt end.

In some aspects, an Addamer can comprise a hairpin that comprises an aptamer sequence, a first IISRE sequence, an N-mer sequence, an at least second IISRE sequence and an MCS sequence. In some aspects, an Addamer can comprise a hairpin that comprises an aptamer sequence, followed by a first IISRE sequence, followed by an N-mer sequence, followed by an at least second IISRE sequence, followed by an MCS sequence. An exemplary schematic of the preceding Addamer is shown in FIG. 2 as Addamer Design #7. In the non-limiting example of Addamer Design #7 shown in FIG. 2, the aptamer sequence is a thrombin aptamer sequence, the first IISRE sequence is an IISRE sequence that when cleaved creates a 4 nucleotide long 5′ overhang, the N-mer sequence is a 3-mer sequence, and the at least second IISRE sequence is an IISRE that when cleaved creates a blunt end.

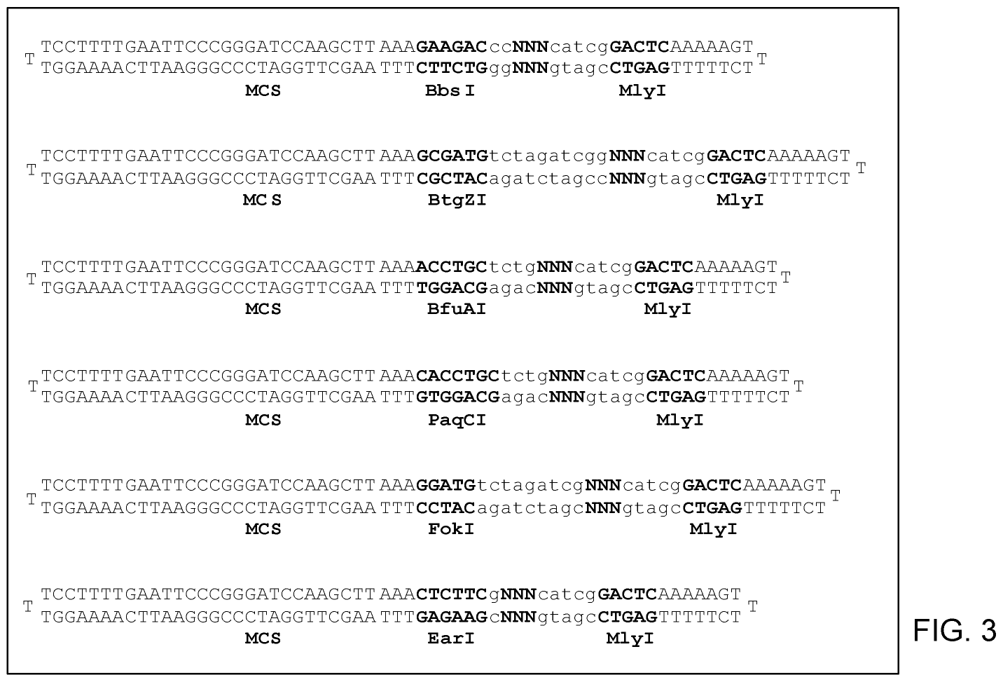

FIG. 3 depicts the sequences of 6 non-limiting examples of Addamers. Each of the Addamers shown in FIG. 3 are capped with simple hairpin turns. Each of the Addamers shown in FIG. 3 comprise an MCS sequence, a first IISRE sequence, an N-mer sequence (denoted with “N”) and a second IISRE sequence. Specifically, the N-mer sequences shown in Addamers of FIG. 3 are 3-mer sequences and the IISRE sequences are selected from BbsI, MlyI, BtgZI, BfuAI, PaqCI, FokI and EarI.

When two IISRE sequences are included adjacent to each other in an Addamer, these IISRE sequences can be called “Nested IISRE sequences” or “Nested IISRE sites”. In some aspects, nested IISRE sequences can comprise two IISRE sequences that are directly adjacent to one another. In some aspects, nested IISRE sequences can comprise two IISRE sequences that are adjacent to one another but separated by about 1 to about 10 nucleotides.

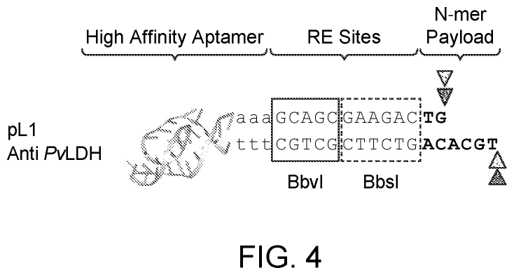

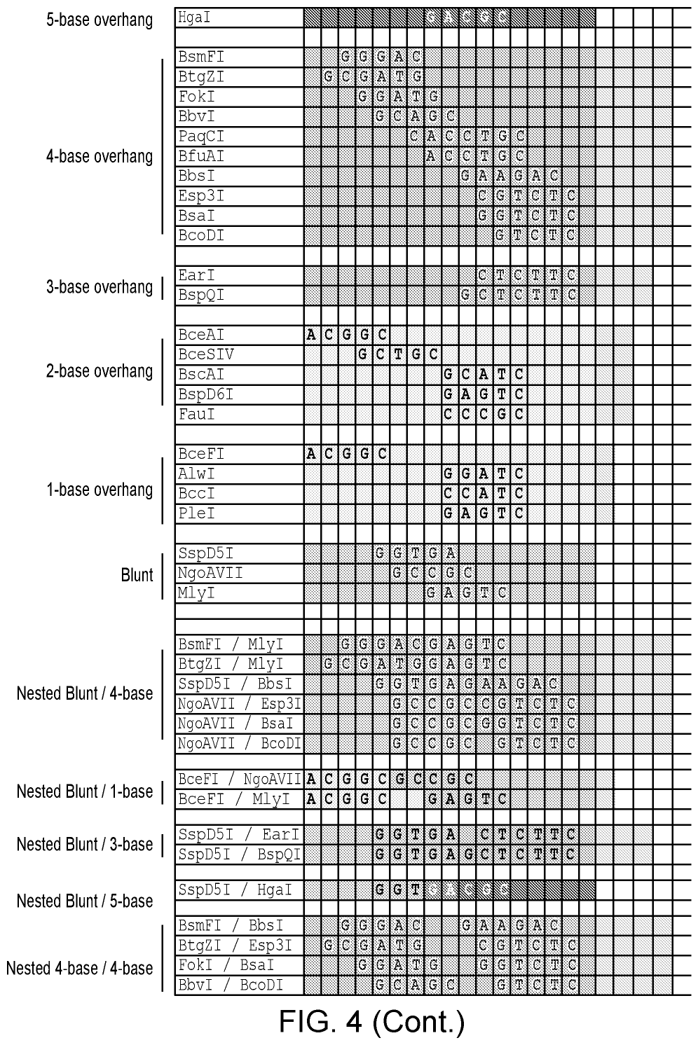

Without wishing to be bound by theory, it is possible to nest IISRE sites because some have cut sites sufficiently spaced from their recognition site as to fit other IISRE sites between the first site and the payload (see FIG. 4). Accordingly, in some aspects an Addamer can comprise a unique blunt cutter sites and a 4-base overhang sites on either side of the payload (N-mer sequence). Without wishing to be bound by theory, this significantly reduces the number of Addamer reagents needed to carry out routine nucleic acid generation.

Without wishing to be bound by theory, the inclusion of Nested IISRE sequence in an Addamer provides several options to cut at the same position with two distinct sites in the methods of the present disclosure. The option to cut at the same position with two distinct sites can reduce the number of distinct Addamers needed in a library (see below) for general nucleic acid synthesis. FIG. 4 shows non-limiting examples of nested IISRE sites, including nested BbvI and BbsI sites. FIG. 4 also shows three non-limiting examples of aptamer sequences that can be including in the hairpins of Addamers described herein.

In some aspects, Addamers can comprise any element known in the art to facilitate cloning, including but not limited to cognate sequences for amplification primers. Without wishing to be bound by theory, the inclusion of cognate sequences for amplification primers in an Addamer can allow for the recovery of specific Addamer designs for clonal propagation.

In some aspects, Addamers can comprise any element known in the art to facilitate large-scale production of the Addamer by fermentation in plasmids or bacteriophage. Such elements include, but are not limited to, sequences corresponding to DNAzyme scars and/or sequences that facilitate smooth folding of an Addamer after excision using certain DNAzymes (see e.g. Praetorius et al., Nature, 2017, 552, 84-87, incorporated herein by reference in its entirety).

### Addamer Libraries

The present disclosure provides Addamer libraries comprising a plurality of Addamers, wherein the plurality of Addamers comprises one or more different Addamer species (i.e. Addamers with unique sequences).

The present disclosure provides a 3-mer Addamer library comprising a plurality of Addamers, wherein the plurality of Addamers comprises at least 64 different Addamer species, wherein each of the Addamer species comprises one of the 64 possible 3-mer sequences that can be created with Adenine, Cytosine, Guanine and Thymine (4×4×4=64).

The present disclosure provides a 4-mer Addamer library comprising a plurality of Addamers, wherein the plurality of Addamers comprises at least 256 different Addamer species, wherein each of the Addamer species comprises one of the 256 possible 4-mer sequences that can be created with Adenine, Cytosine, Guanine and Thymine (4×4×4×4=256).

The present disclosure provides a 5-mer Addamer library comprising a plurality of Addamers, wherein the plurality of Addamers comprises at least 1,024 different Addamer species, wherein each of the Addamer species comprises one of the 1,024 possible 5-mer sequences that can be created with Adenine, Cytosine, Guanine and Thymine (4×4×4×4×4=1,024).

The present disclosure provides a 6-mer Addamer library comprising a plurality of Addamers, wherein the plurality of Addamers comprises at least 256 different Addamer species, wherein each of the Addamer species comprises one of the 256 possible 6-mer sequences that can be created with Adenine, Cytosine, Guanine and Thymine (4×4×4×4×4×4=4,096).

### Methods of Producing Addamers

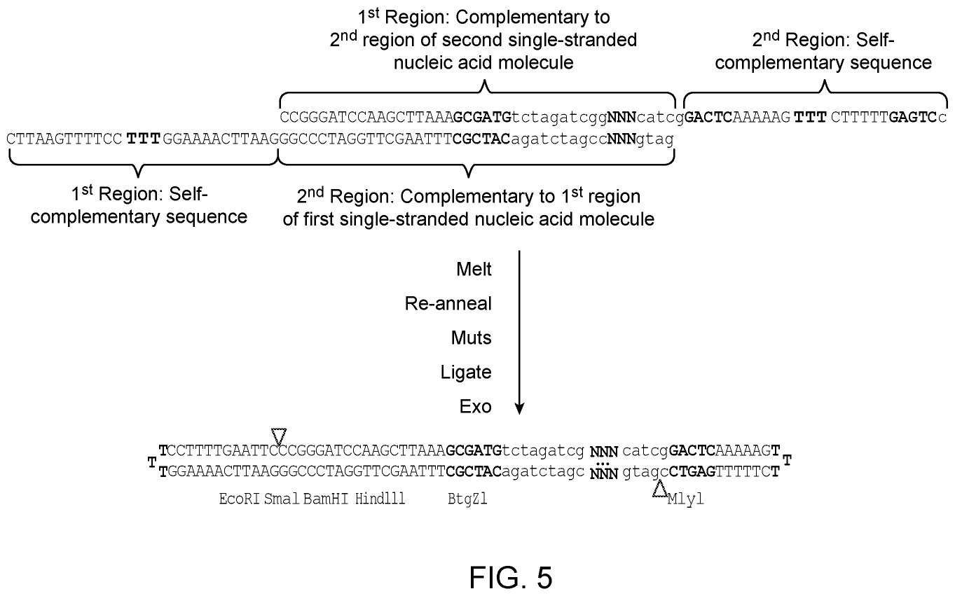

Addamers described herein can be produced using chemically synthesized nucleic acids in methods that are schematically shown in FIG. 5. In this method, a first single-stranded nucleic acid molecule and a second single-stranded nucleic acid molecule are chemically synthesized (e.g. using phosphoramidite synthesis). The first single-stranded nucleic acid molecule comprises a first region that is complementary to a second region on the second single-stranded nucleic acid molecule and the second region that is self-complementary, and the second single-stranded nucleic acid molecule comprises a first region that is self-complementary and a second region that is complementary to a first region on the first single-stranded nucleic acid molecule, as shown in the top panel of FIG. 5. The first single-stranded nucleic acid molecule and the second single-stranded nucleic acid molecule are then hybridized together to produce a partially double-stranded nucleic acid molecule. The partially double-stranded nucleic acid molecule can then be optionally contacted with the enzyme MutS, which binds to mismatched bases and exposes DNA to exonuclease digestion. The partially double-stranded nucleic acid molecule can then be contacted with a ligase enzyme to form the double-stranded Addamer structure capped at both ends by hairpins. Following contact with the ligase enzyme, the product can be contacted with T7 exonuclease to purify and enrich for properly formed Addamers.

Without wishing to be bound by theory, the preceding method of producing Aptamers using chemically synthesized single-stranded nucleic acid molecules allows for a rapid turn-around time for testing new Addamer designs. Without wishing to be bound by theory, the Addamers produced using the preceding method can then be used as templates for large-scale production of said Addamers using methods that do not require phosphoramidite synthesis, including, but not limited to, clonal production of the Addamer using a plasmid or bacteriophage.

Accordingly, the present disclosure provides methods of producing the Addamers described herein, the methods comprising: a) providing a first single-stranded nucleic acid molecule and a second single-stranded nucleic acid molecule, wherein the sequences of the first single-stranded nucleic acid molecule and second single-stranded nucleic acid molecule comprise portions of the Addamer that is to be produced, wherein the first single stranded nucleic acid molecule comprises a first region that is complementary to a second region on the second single-stranded nucleic acid molecule and the second region that is self-complementary, and the second single-stranded nucleic acid molecule comprises a first region that is self-complementary and a second region that is complementary to a first region on the first single-stranded nucleic acid molecule; b) hybridizing the first single-stranded nucleic acid and the second single-stranded nucleic acid to produce a partially double-stranded nucleic acid molecule; and c) contacting the partially double-stranded nucleic acid molecule with a ligase enzyme to form a double-stranded Addamer structure capped at both ends by hairpins.

Accordingly, the present disclosure provides methods of producing the Addamers described herein, the methods comprising: a) chemically synthesizing a first single-stranded nucleic acid molecule and a second single-stranded nucleic acid molecule, wherein the sequences of the first single-stranded nucleic acid molecule and second single-stranded nucleic acid molecule comprise portions of the Addamer that is to be produced, wherein the first single stranded nucleic acid molecule comprises a first region that is complementary to a second region on the second single-stranded nucleic acid molecule and the second region that is self-complementary, and the second single-stranded nucleic acid molecule comprises a first region that is self-complementary and a second region that is complementary to a first region on the first single-stranded nucleic acid molecule; b) hybridizing the first single-stranded nucleic acid and the second single-stranded nucleic acid to produce a partially double-stranded nucleic acid molecule; and c) contacting the partially double-stranded nucleic acid molecule with a ligase enzyme to form a double-stranded Addamer structure capped at both ends by hairpins.

In some aspects, the preceding methods can further comprise treating the products of step (c) with an exonuclease, thereby purifying properly ligated Addamers.

In some aspects, the preceding methods can further comprise after step (b) and before step (c), contacting the partially double-stranded nucleic acid molecule with a MutS enzyme.

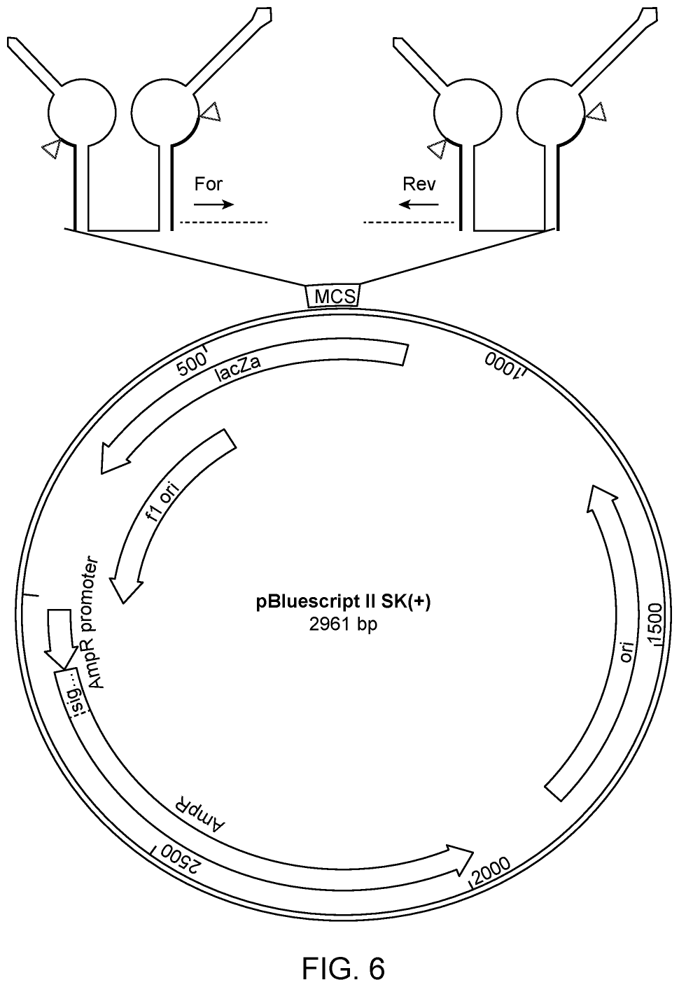

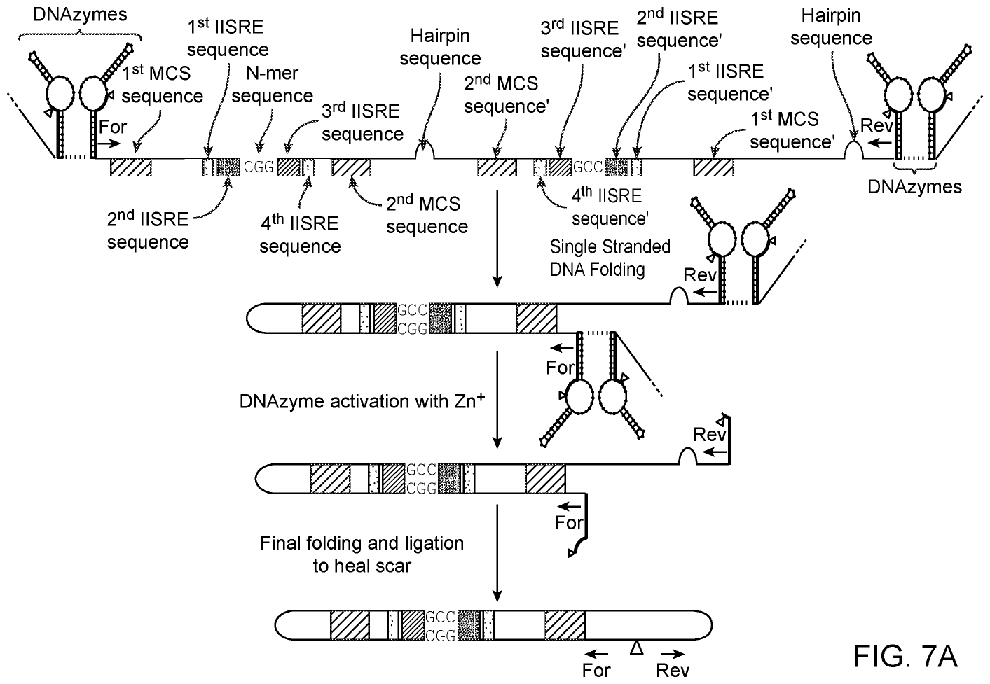

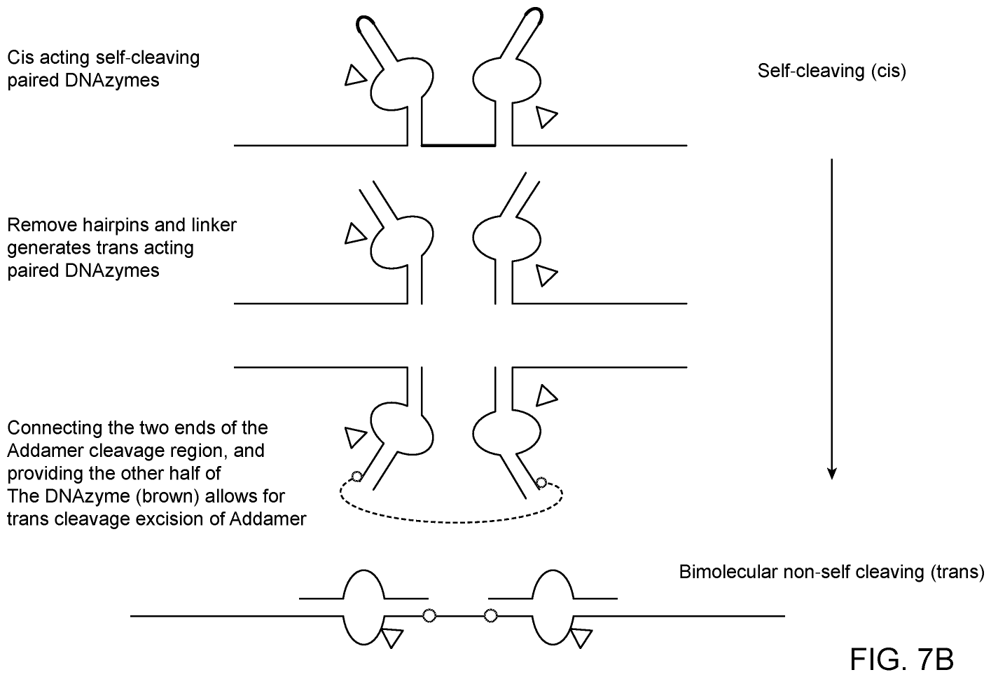

Addamers described herein can be produced using phagemid-based methods that are schematically shown in FIG. 6 and FIG. 7A (see also Praetorius et al., Nature, 2017, 552, 84-87 and US Patent Application Publication No. US20190203242A1, each of which are incorporated herein by reference in their entireties for all purposes). In this method, an Addamer sequence is cloned into a phagemid (e.g. pBluescript or any other phagemid known in the art) as shown in FIG. 6. The Addamer sequence is flanked on both sides by one or more DNAzymes that can be selectively activated (e.g. by contacting the DNAzymes with Zn²⁺) as shown in FIG. 6 and the top panel of FIG. 7A. The phagemid is then converted to an M13 packaged bacteriophage using a helper phage by methods that are well known to the skilled artisan. The M13 packaged bacteriophage produces single-stranded DNA that comprises the Addamer sequence and flanked DNAzymes, as shown in the top panel of FIG. 7A. The single-stranded DNA that is produced by the bacteriophage is purified and allowed to fold to produce DNAzyme structures and the majority portion of the double-stranded Addamer sequence, as shown in the second panel from the top of FIG. 7A. The DNAzymes are then activated (e.g. by contacting the DNAzymes with Zn²⁺), excising the Addamer sequence from the single-stranded DNA produced by the bacteriophage, as shown in the second panel from the bottom of FIG. 7A. After DNAzyme cleavage, the Addamer is allowed to fold and is then contacted by a ligase to close the Addamer, as shown in the bottom panel of FIG. 7A. Optionally, non-ligated or improperly formed Addamers can be removed by treating the products of the ligation reaction with T7 exonuclease. The exemplary Addamer depicted in FIG. 7A comprises a 1^{st} MCS sequence, followed by a 1^{st} IISRE sequence, followed by a 2^{nd} IISRE sequence, followed by an N-mer sequence, followed by a 3^{rd} IISRE sequence, followed by a 4^{th} IISRE sequence, followed by a 2^{nd} MCS sequence. The apostrophes in FIG. 7A denote a reverse complementary sequence such that the Addamer is self-complementary and forms a double-stranded structure capped at both ends with hairpin domains.

Alternatively, similar bacteriophage DNA preparations can be treated with trans-acting DNAzyme elements that are hybridized to M13 DNA, activated by Zn2+, which then excise the Addamers in a non-self-cleaving manner. As would be appreciated by the skilled artisan DNAzymes have been shown to act as bimolecular reagents (see Gu, et al., Journal of the American Chemical Society, 2013, 135, 24, 9121-9129, the contents of which are incorporated herein by reference in their entireties). Thus, in some aspects, Addamer excision can be achieved with hemi-DNAzyme sequence being provided in trans, hybridized to bacteriophage preparations and activated with Zn²⁺ (see FIG. 7B).

Accordingly, the present disclosure provides methods of producing the Addamers described herein, the methods comprising: a) cloning the Addamer sequence into a phagemid such that the Addamer sequence is flanked on both sides by one or more DNAzymes that can be selectively activated; b) converting the phagemid into packaged bacteriophage using a helper phage, wherein the packaged bacteriophage produces single-stranded DNA comprising the Addamer sequence flanked on both sides by one or more DNAzymes; c) purifying the single-stranded DNA produced by the packaged bacteriophage; d) allowing the purified single-stranded DNA to fold to produce the one or more DNAzyme structure and a majority portion of the double-stranded Addamer sequence; e) activating the one or more DNAzymes, thereby excising the Addamer sequence from the single-stranded DNA produced by the packaged bacteriophage; f) contacting the excised Addamer with a ligase enzyme.

Accordingly, the present disclosure provides methods of producing the Addamers described herein, the methods comprising: a) cloning the Addamer sequence into a phagemid such that the Addamer sequence is flanked on both sides by one or more first portions of a DNAzyme that can be selectively activated; b) converting the phagemid into packaged bacteriophage using a helper phage, wherein the packaged bacteriophage produces single-stranded DNA comprising the Addamer sequence flanked on both sides by one or more DNAzymes; c) purifying the single-stranded DNA produced by the packaged bacteriophage; d) allowing the purified single-stranded DNA to fold to produce a majority portion of the double-stranded Addamer sequence; e) contacting the purified single-stranded DNA with one or more oligonucleotides comprising the second portions of the DNAzymes, wherein the one or more oligonucleotides hybridize to the purified single-stranded DNA such that one or more complete DNAzymes are formed; f) activating the one or more DNAzymes, thereby excising the Addamer sequence from the single-stranded DNA produced by the packaged bacteriophage; and g) contacting the excised Addamer with a ligase enzyme.

Accordingly, the present disclosure provides methods of producing the Addamers described herein, the methods comprising: a) cloning the Addamer sequence into a phagemid such that the Addamer sequence is flanked on both sides by one or more first portions of a DNAzyme that can be selectively activated; b) converting the phagemid into packaged bacteriophage using a helper phage, wherein the packaged bacteriophage produces single-stranded DNA comprising the Addamer sequence flanked on both sides by one or more DNAzymes; c) purifying the single-stranded DNA produced by the packaged bacteriophage; d) contacting the purified single-stranded DNA with one or more oligonucleotides comprising the second portions of the DNAzymes, wherein the one or more oligonucleotides hybridize to the purified single-stranded DNA such that one or more complete DNAzymes are formed; e) allowing products of step (d) to fold to produce a majority portion of the double-stranded Addamer sequence; (f) activating the one or more DNAzymes, thereby excising the Addamer sequence from the single-stranded DNA produced by the packaged bacteriophage; and g) contacting the excised Addamer with a ligase enzyme.

In some aspects, the preceding methods can further comprise, after treating the excised Addamer with a ligase enzyme, treating said products with at least one exonuclease, thereby purifying properly ligated Addamers.

Addamers described herein can be produced using plasmid-based methods. In the plasmid-based methods, Addamers are cloned into a plasmid that can be propagated and purified at a large-scale (e.g. in bacteria and/or yeast). The purified plasmids comprising the Addamer sequences can then be treated with one or more nickase enzymes (e.g. Cas9 Nickase with appropriate guide RNAs) and/or restriction endonuclease enzymes to excise the Addamer sequences from the plasmid to yield double-stranded Addamers with DNA flaps at each end. The double-stranded Addamers with DNA flaps can then be treated with one or more ligase enzymes to produce double-stranded Addamers capped at both ends by a hairpin structure.

Accordingly, the present disclosure provides methods of producing the Addamers described herein, the methods comprising: a) cloning the Addamer sequence into a plasmid; b) propagating the plasmid in a suitable host organism; c) purifying the plasmid from the host organism; d) treating the purified plasmid with one or more of a nickase enzyme and a restriction endonuclease enzyme to excise the Addamer sequences from the plasmid; and e) contacting the excised Addamer sequences with ligase to produce double-stranded Addamer structures capped at both ends by a hairpin structure.

The methods for producing Addamers described herein can be used to generate Addamer libraries, including, but not limited to 3-mer Addamer libraries, 4-mer Addamer libraries, 5-mer Addamer libraries and 6-mer Addamer libraries.

In some aspects, a 6-mer Addamer library may be generated from a 3-mer Addamer library. Without wishing to be bound by theory, although the total number of possible hexamers is large, the hexamer payload is an extremely useful Addamer type for oligonucleotide synthesis. Additionally, it would allow routine switching between IISREs sites encoded in left side versus right side trimer elements. In a non-limiting example, an initial library Initially, libraries of 64 element trimer Addamers are generated using conventional phosphoramidite synthesis either individually or as complete sets in DNA microarray pools. Pools of Addamer trimer libraries are cloned en masse into bacteriophage or amplified in vivo. Left side and right side Addamer trimer libraries are prepared and digested to generate blunt ends at the payload site. The right and left side libraries are then ligated to form a large pool of Addamers. Un-ligated material is digested away by exonuclease. The remaining intact Addamers are then used as templates to amplify specific hexamer Addamers by PCR. Each independent hexamer PCR product can then be cloned into bacteriophage for production at the appropriate scale and can also be stored for future use.

### Nucleic Acid Synthesis Methods Using Addamers of the Present Disclosure

The Addamers described herein can be used in the methods described herein to synthesize a nucleic acid molecule comprising any target nucleic acid sequence.

In some aspects, a target nucleic acid sequence can be at least about 100, or at least about 200, or at least about 300, or at least about 500, or at least about 600, or at least about 700, or at least about 800, or at least about 900, or at least about 1000, or at least about 1500, or at least about 2000, or at least about 2500, or at least about 3000, or at least about 3500, or at least about 4000, or at least about 4500, or at least about 5000 nucleotides in length. In some aspects, the target double-stranded nucleic acid can comprise at least one homopolymeric sequence.

In some aspects, the target nucleic acid sequence can comprise at least one homopolymeric sequence. As used herein, the term homopolymeric sequence is used to refer to any type of repeating nucleic acid sequence, including, but not limited to, repeats of single nucleotides or repeats of small motifs. In some aspects, a homopolymeric sequence can be at least about 10 nucleotides, or at least about 20 nucleotides, or at least about 30 nucleotides, or at least about 40 nucleotides, or at least about 50 nucleotides, or at least about 60 nucleotides, or at least about 70 nucleotides, or at least about 80 nucleotides, or at least about 90 nucleotides, or at least about 100 nucleotides in length.

In some aspects, the target nucleic acid sequence can have a GC content of at least about 10%, or at least about 20%, or at least about 50%, or at least about.

As part of the synthesis methods of a present disclosure, one or more Addamers can be immobilized to a solid support. The solid support can be any solid support known in the art, including, but not limited to at least one bead. In some aspects, the at least one bead can comprise polyacrylamide, polystyrene, agarose or any combination thereof. In some aspects, the at least one bead can be magnetic. In some aspects, the solid support comprises a well or chamber. In some aspects, a solid support can comprise a plurality of wells or chambers. In some aspects, the plurality of wells comprises a multi-well plate. In some aspects, a solid support can comprise glass. In some aspects, a solid support can comprise a glass slide. In some aspects, a solid support can comprise quartz. In some aspects, a solid support can comprise a quartz slide. In some aspects, a solid support can comprise polystyrene. In some aspects, a solid support can comprise a polystyrene slide. In some aspects, a solid support can comprise a coating, wherein the coating prevents non-specific binding of unwanted proteins, unwanted nucleic acids or other unwanted biomolecules. In some aspects, a coating can comprise polyethylene glycol (PEG). In some aspects, a coating can comprise triethylene glycol (TEG).

In some aspects wherein an Addamer comprises a hairpin that comprises an aptamer sequence, the Addamer can be immobilized to a solid support via binding to the aptamer sequence. That is, the solid support can comprise at least one moiety that binds to the aptamer sequence on the Addamer. Accordingly, in a non-limiting example wherein an Addamer comprises a hairpin that comprises one of the aptamer sequences put forth in Table 2, the solid support can comprise the corresponding ligand listed in Table 2.

In some aspects wherein an Addamer comprises an MCS sequence, the Addamer can be immobilized to a solid support by a method comprising: a) contacting the Addamer with at least one corresponding restriction endonuclease to cleave the MCS sequence, thereby producing a 5′ overhang or a 3′ overhang; and b) hybridizing the 5′ overhang or 3′ overhang to a complementary single-stranded nucleic acid molecule on the solid support, thereby immobilizing the Addamer to the solid support. The preceding method can further comprise contacting the Addamer hybridized to the complementary single-stranded nucleic acid molecule on the solid support with a ligase, thereby ligating the Addamer and the complementary single-stranded nucleic acid molecule on the solid support.

In some aspects wherein an Addamer comprises an MCS sequence, the Addamer can be immobilized to a solid support by a method comprising: a) contacting the Addamer with at least one corresponding restriction endonuclease to cleave the MCS sequence, thereby producing a blunt end; and b) ligating the blunt end of the Addamer to a nucleic acid molecule located on the solid support, thereby immobilizing the Addamer to the solid support.

In some aspects, an Addamer that has been attached to a solid support can be referred to herein as an “attachment stud”.

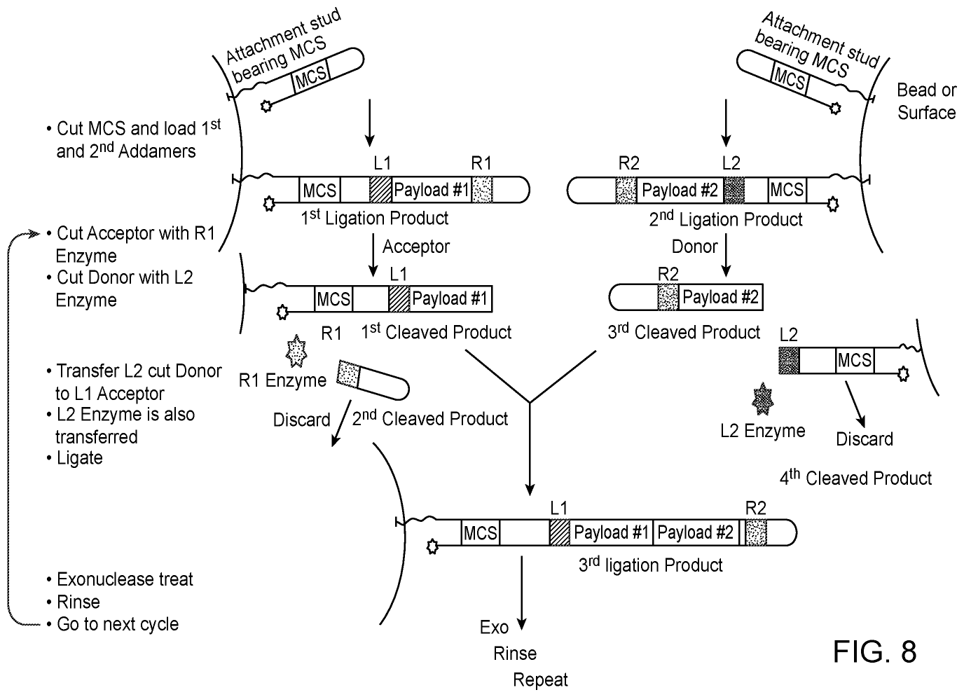

A schematic overview of the nucleic acid synthesis methods of the present disclosure is shown in FIG. 8.

In the first step of the method, an Addamer immobilized onto a solid support (denoted as a bead or surface in FIG. 8) is provided. This Addamer is herein referred to as an attachment stud and is connected at one end to the solid support using any of the methods described above and is capped at the other end with a hairpin. The attachment stud also comprises an MCS sequence. In the next step of the method, the attachment stud is contacted with a restriction endonuclease that cleaves the MCS sequence, thereby creating a 3′ overhang, a 5′ overhang or a blunt end. In the next step of the method, a first Addamer comprising an MCS sequence, a first IISRE sequence (denoted “L1” in FIG. 8), a first N-mer sequence (referred to as “Payload #1” in FIG. 8) and a second IISRE sequence (denoted “R1” in FIG. 8) is contacted with a restriction endonuclease that cleaves the MCS sequence, thereby creating a 3′ overhang, a 5′ overhang or a blunt end.

In the next step of the method, the cleaved first Addamer is ligated to the cleaved attachment stud by contacting the cleaved attachment stud, the cleaved Addamer and a ligase enzyme, thereby creating a first ligation product that is immobilized to the solid support and that comprises the MCS sequence, the first IISRE sequence, the Payload #1 sequence and the second IISRE sequence (see left side of FIG. 8). The first ligation product is then treated with exonuclease to remove any non-ligated attachment studs and/or first Addamers.

The steps described above are then repeated with another Addamer immobilized onto a solid support and a second Addamer comprising an MCS sequence, a third IISRE sequence (denoted “R2” in FIG. 8), a second N-mer sequence (referred to as “Payload #2” in FIG. 8) and a fourth IISRE sequence (denoted “L2” in FIG. 8) to produce a second ligation product that is immobilized to a solid support and that comprises an MCS sequence, the third IISRE sequence, the Payload #2 sequence and the fourth IISRE sequence (see right side of FIG. 8).

In the next step of the method, the 1^{st} ligation product is contacted with a IISRE (denoted “R1 enzyme” in FIG. 8) that cleaves the second IISRE sequence (R1), thereby creating a 3′ overhang, a 5′ overhang or a blunt end, thereby creating: a) a 1^{st} cleaved product that is immobilized to the solid support and that comprises the MCS sequence, the first IISRE sequence (R1), the Payload #1 sequence and a 3′ overhang, a 5′ overhang or a blunt end; and b) a 2^{nd} cleaved product comprising the second IISRE sequence (R1). The 2^{nd} cleaved product is then discarded by washing.

In the next step of the method, the 2^{nd} ligation product is contacted with a IISRE (denoted “L2 enzyme” in FIG. 8) that claves the fourth IISRE sequence (L2), thereby creating a 3′ overhang, a 5′ overhang or a blunt end, thereby creating: a) a 3^{rd} cleaved product that is released into solution and that comprises a hairpin at one end, the 3^{rd} IISRE sequence (R2), the Payload #2 sequence and a 3′ overhang, a 5′ overhang or a blunt end; and b) a 4^{th} cleaved product that is immobilized to the solid support and that comprises the MCS sequence and the fourth IISRE sequence (L2).

In the next step, the 1^{st} cleaved product and the 3^{rd} cleaved product are ligated together by contacting the 1^{st} cleaved product, the 3^{rd} cleaved product and a ligase enzyme (e.g. the solution comprising the 3^{rd} cleaved product is transferred to the solution comprising the 1^{st} cleaved product immobilized to the solid surface and a ligase enzyme is added to the solution), thereby creating a 3^{rd} ligation product that is immobilized to a solid surface and that comprises an MCS sequence, the first IISRE sequence (L1), the Payload #1 sequence, the Payload #2 sequence and the third IISRE sequence (R2). This ligation reaction is then treated with exonuclease to remove any non-ligated 1^{st} Cleaved Products and/or 3^{rd} Cleaved Products.

The steps described above can be repeated until a target nucleic acid sequence is synthesized.

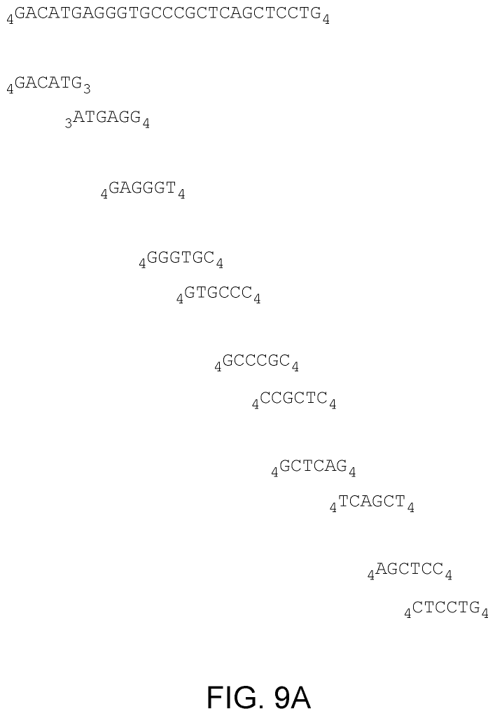

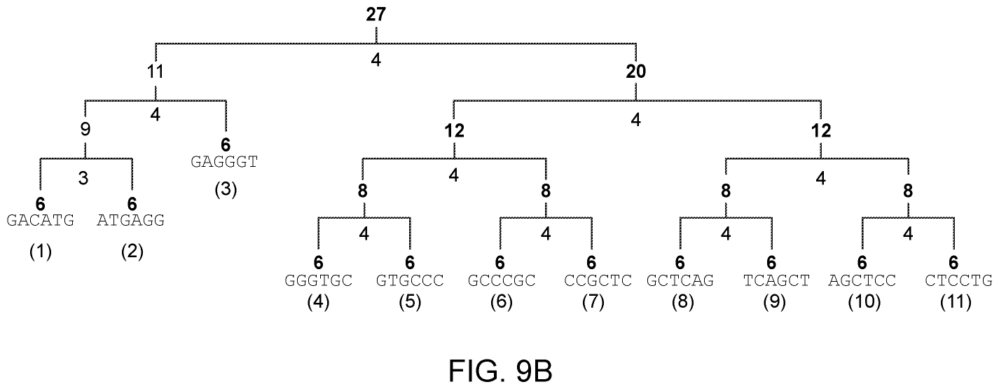

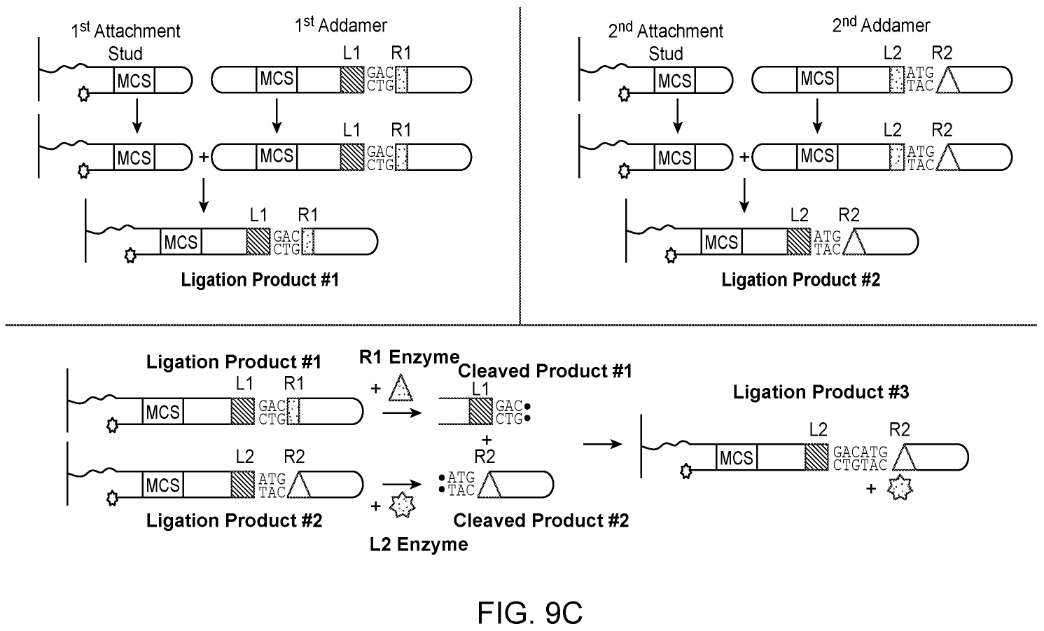

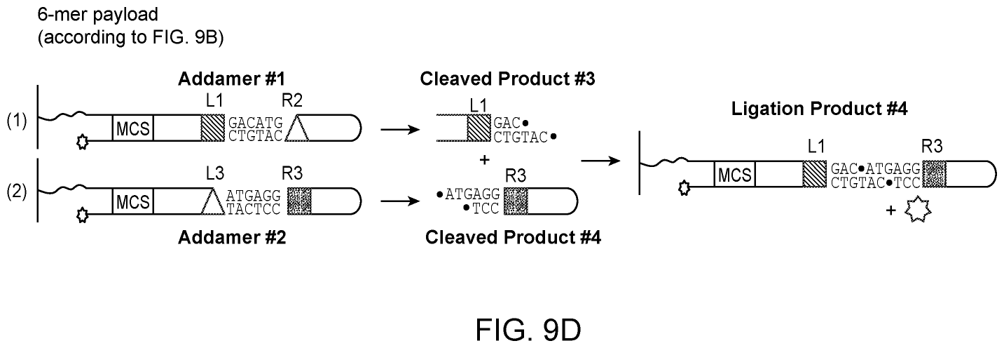

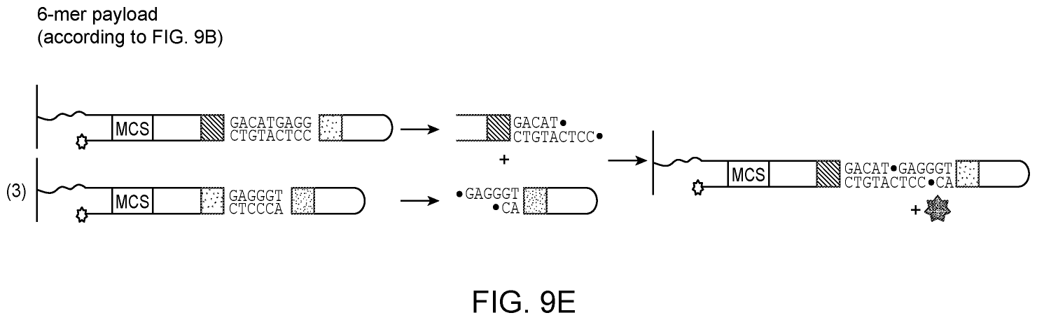

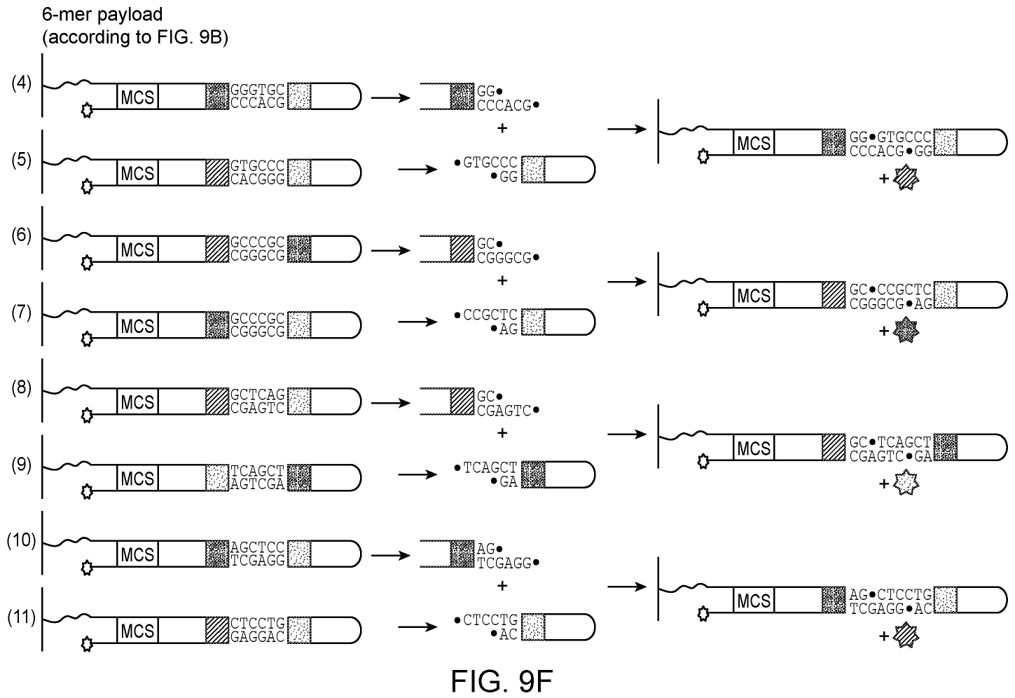

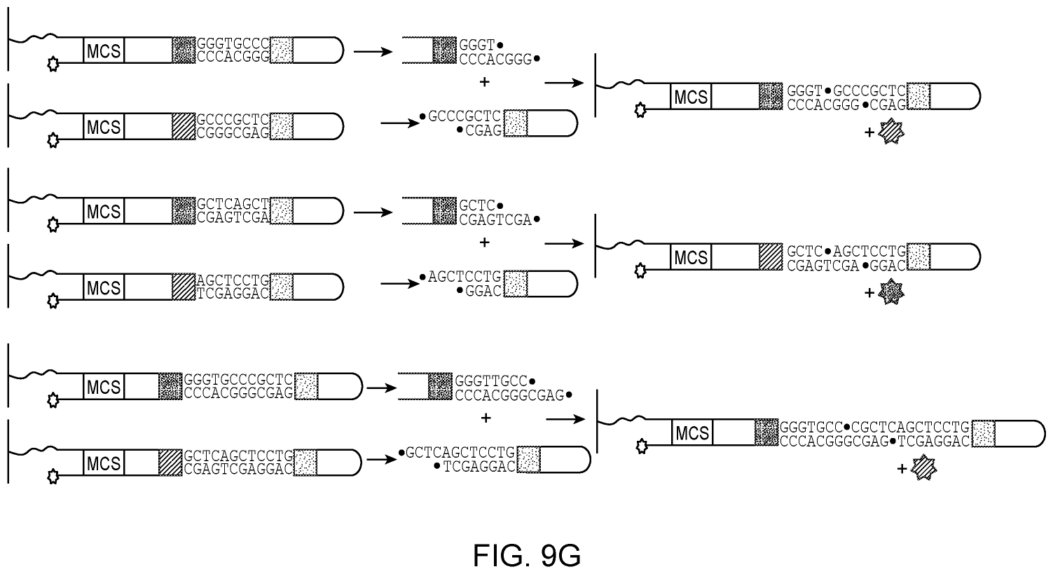

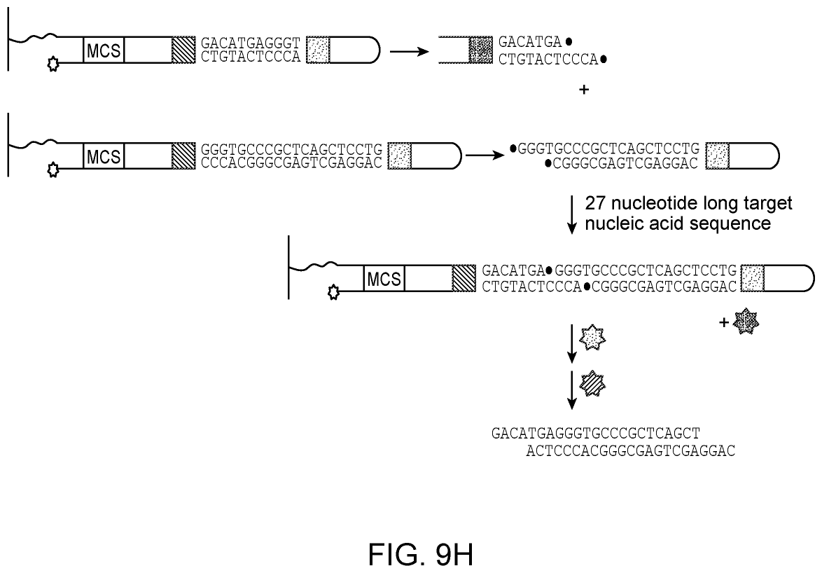

A schematic overview of the synthesis of an exemplary 27-nucleotide long target nucleic acid sequence is shown in FIG. 9A-9H. The sequence to be synthesized is shown at the top of FIG. 9A. The sequence is subdivided into eleven, 6-mer fragments that overlap by either 3 nucleotides or 4 nucleotides that are to be incorporated into Addamers that are to be ligated together to synthesize the target nucleic acid sequence. FIG. 9B shows an assembly tree for the exemplary target nucleic acid sequence that maps the order in which the Addamers comprising the 6-mer fragments are to be ligated to efficiently synthesize the target nucleic acid sequence. While there are several different ways to traverse the assembly and placement of odd versus even overhangs, the assembly order is to be dictated by the compatibility of IISRE enzyme sites with the sequences to be generated. In FIG. 9B the numbered 6-mers (1)-(11) correspond to the numbered 6-mers in FIGS. 9C-9H. The numbers at each node of the tree correspond to the payload length at each step of the assembly. The ‘4’ and ‘3’ indicate the length of the overhang used. The length of a resulting payload sequence is, length=a+b−n, where ‘a’ and ‘b’ are the lengths of the input payloads and ‘n’ is the length of the overhang.

The first step of the synthesis of the target nucleic acid sequence is shown in FIG. 9C, which depicts the loading of an Addamer comprising an 3-mer sequence of GAC and an Addamer comprising an 3-mer sequence of ATC to form an Addamer comprising a GACATG 6-mer, which is 6-mer #1 in FIG. 9B. To produce the GACATG hexamer, a first attachment stud comprising an MCS sequence and a first Addamer comprising an MCS sequence, a first IISRE sequence (denoted “L1” in FIG. 9C), a 3-mer sequence comprising the sequence GAC, and a second IISRE sequence (denoted “R1” in FIG. 9C) are contacted with one or more restriction endonucleases to cleave the MCS sequences, thereby creating complementary overhangs. These complementary overhangs are then hybridized and the Addamer and the attachment stud are ligated together by contacting the hybridized complex with a ligase enzyme to yield Ligation Product #1 that is immobilized to the solid surface and that comprises the MCS sequence, the first IISRE sequence (L1), the 3-mer sequence GAC, and the second IISRE sequence (R1). The same process can be repeated with a second attachment stud comprising an MCS sequence and a second Addamer comprising an MCS sequence, a third IISRE sequence (denoted “L2” in FIG. 9C), the 3-mer sequencing comprising the sequence ATG, and a fourth IISRE sequence (denoted “R2” in FIG. 9C) to yield Ligation Product #2 that is immobilized to the solid surface and that comprises the MCS sequence, the third IISRE sequence (L2), the 3-mer sequence ATG and the fourth IISRE sequence (R2). Next, Ligation Product #1 is contacted with a IISRE (denoted “R1 enzyme” in FIG. 9C) that cleaves the second IISRE sequence (R1), thereby producing Cleaved Product #1 that is immobilized to the solid surfaces and that comprises the MCS sequence, the first IISRE sequence (L1) and the 3-mer sequence GAC followed by a blunt end. Similarly, Ligation Product #2 is contacted with a IISRE (denoted “L2 enzyme” in FIG. 9C) that cleaves the third IISRE sequence (L2), thereby producing Cleaved Product #2 that is released into solution and that comprises the a blunt end, the 3-mer sequence ATG and the fourth IISRE sequence (R2). Cleaved Product #1 and Cleaved Product #2 are then ligated together using a ligase enzyme to yield Ligation Product #3 that is immobilized to the solid support and that comprises the MCS sequence, the first IISRE sequence (L1), the 6-mer sequence GACATG and the fourth IISRE sequence (R2). These products can optionally be treated with exonuclease to remove any non-ligated Cleaved Product #1 and/or Cleaved Product #2. The steps described in this paragraph can be repeated with additional Addamers comprising different 3-mer sequences to generate the Addamers comprising 6-mer sequences #2-#11 shown in FIG. 9B.

The method continues in FIG. 9D, which shows the ligation of Addamers comprising 6-mer Sequence #1 and 6-mer Sequence #2 (see FIG. 9B). Addamer #1 is immobilized to a solid surface and comprises an MCS sequence, the first IISRE site (L1) from FIG. 9C, the 6-mer sequence GACATG (6-mer sequence #1 from FIG. 9B), and the fourth IISRE sequence (R2) from FIG. 9C. Addamer #2 is immobilized to a solid surface and comprises an MCS sequence, a fifth IISRE site (denoted “L3” in FIG. 9D), the 6-mer sequence ATGAGG (6-mer sequence #2 from FIG. 9B) and a sixth IISRE site (denoted “R3” in FIG. 9D). Addamer #1 is contacted with a IISRE that cleaves the fourth IISRE site (R2) to produce a single-stranded overhang in the N-mer sequence, thereby producing Cleaved Product #3 that is immobilized to the solid surface and that comprises the MCS sequence, the first IISRE sequence (L1), and the N-mer sequence with a single-stranded overhang. Addamer #2 is contacted with a IISRE that claves the fifth IISRE site (L3) to produce a single-stranded overhang in the N-mer sequence, thereby producing Cleaved Product #4 that is released into solution and the comprises the N-mer sequence with a single-stranded overhang and the sixth IISRE sequence (R3). Cleaved Product #3 and Cleaved Product #4 are then ligated together using a ligase enzyme to yield ligation Product #4 that is immobilized to the solid surface and that comprises the MCS sequence, the first IISRE sequence (L1), the N-mer sequence GACATGAGG (the first nine nucleotides in the target nucleic acid sequence to be synthesized) and the sixth IISRE sequence (R3). Ligation Product #4 can optionally be treated with exonuclease to remove any non-ligated Cleaved Product #1 and/or Cleaved Product #2.

The method continues in FIG. 9E, where Ligation Product #4 and an Addamer that comprises 6-mer Sequence #3 (see FIG. 9B) are treated with corresponding IISREs to produce cleaved products that are then ligated together to produce an Addamer that is immobilized to the solid surface and that comprises the N-mer sequence GACATGAGGGT (SEQ ID NO: 75), the first 11 nucleotides in the target nucleic acid sequence to be synthesized.

The sequential IISRE digestions and ligations are repeated in FIGS. 9F-9H according the assembly map shown in FIG. 9B until an Addamer comprising an N-mer sequence that corresponds to the 27 nucleotide long target nucleic acid sequence is synthesized. In a final step, the 27 nucleotide long target nucleic acid sequence can be excised from the final synthesized Addamer by treating the Addamer with IISREs that cleave the IISRE sequences that flank the 27 nucleotide long target nucleic acid sequence.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (c)-(f) until the nucleic acid molecule comprising the target nucleic acid sequence has been synthesized.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) with one or more additional Addamers until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (c)-(f) with one or more additional addamers until the nucleic acid molecule comprising the target nucleic acid sequence has been synthesized.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) using the products of step (f) and one or more additional Addamers until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (c)-(f) using the products of step (f) and one or more additional Addamers until the nucleic acid molecule comprising the target nucleic acid sequence has been synthesized.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating any combination of steps (a)-(f) using the products of step (f) and/or one or more additional Addamers until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating any combination of steps (c)-(f) using the products of step (f) and/or one or more additional Addamers until the nucleic acid molecule comprising the target nucleic acid sequence has been synthesized.

In some aspects of the methods of the present disclosure, a ligase enzyme can be a human DNA ligase III (hLig3). As would be appreciated by the skilled artisan, hLig3 exhibits high blunt end ligation efficiency (>60%). In some aspects of the methods of the present disclosure, a ligase enzyme can be a T4 DNA ligase. As would be appreciated by the skilled artisan, T4 DNA ligase exhibits high ligation efficiency of nucleic acid fragments comprising 2, 3 or 4-nucleotide long 3′ or 5′ overhangs (>80%). A ligase enzyme can be any ligase enzyme known in the art.

In some aspects of the methods of the present disclosure, the exonuclease can be T7 exonuclease.

In some aspects of the methods of the present disclosure, the synthesized target nucleic acid sequence has a purity of at least about 80%, or at least about 85%, or at least about 90%, or at least about 95%, or at least about 99%.

In some aspects, the purity of a synthesized target nucleic acid sequence refers to the percentage of the total ligation products that were formed as part of a single ligation reaction, or multiple rounds of ligation reactions, that correspond to the correct/desired ligation product. Without wishing to be bound by theory, the methods of the present disclosure comprising the ligation of nuclei acid molecules produce can produce plurality of ligation products, some of which correspond to the correct/desired ligation product, and some that are undesired (side-reactions, incorrect ligations, etc.). The purity of a ligation product, or a target molecule that is being synthesized, can be expressed as a percentage, which corresponds to the percentage of the total ligation products formed which correspond to the correct/desired ligation product.

### Example 1—Addamer-Based Synthesis

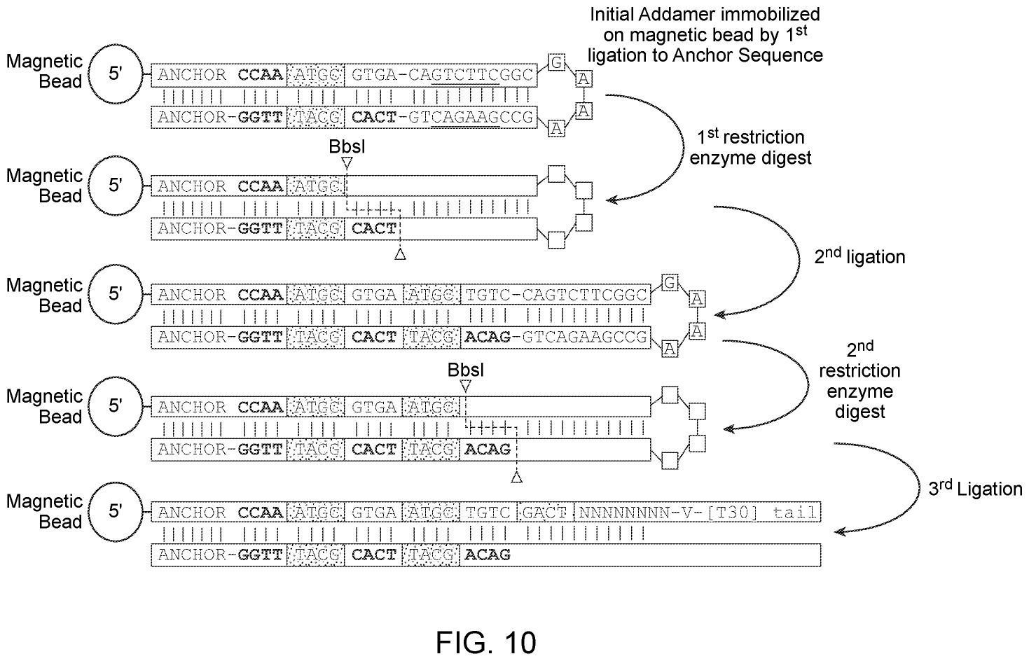

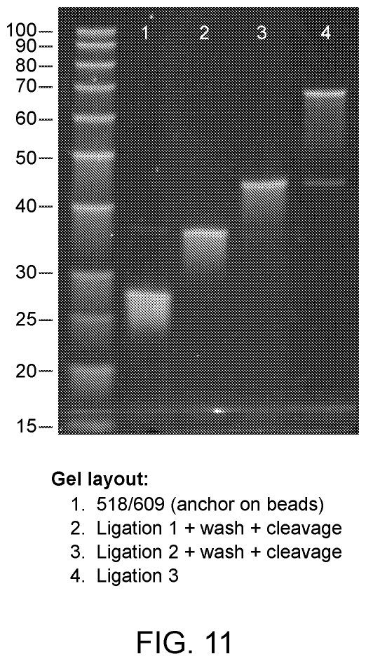

The following is a non-limiting example of the Addamer-based synthesis methods of the present disclosure used to synthesize target nucleic acid molecules. In this example, a series of restriction enzyme digests and subsequent ligations were performed as outlined in FIG. 10. The products of each of the ligation reactions were analyzed using gel electrophoresis. The results of this analysis are shown in FIG. 11. The results shown in FIG. 11 demonstrate that the methods of the present disclosure that comprise iterative restriction enzyme digestions and subsequent ligation reactions using the Addamers of the present disclosure can efficiently synthesize nucleic acid molecules.

What is claimed is:

1. A double-stranded Addamer, wherein the Addamer comprises:
a) a first Type II S restriction endonuclease (IISRE) sequence;
b) an N-mer sequence;
c) an at least second IISRE sequence; and
wherein at least one end of the Addamer comprises a hairpin structure.

2. The Addamer of claim 1, wherein the Addamer comprises a hairpin structure at both ends of the Addamer.

3. The Addamer of any one of the preceding claims, wherein the Addamer comprises:
a) a first IISRE sequence;
b) a second IISRE sequence;
c) an N-mer sequence; and
d) an at least third IISRE sequence.

4. The Addamer of any one of the preceding claims, wherein the Addamer comprises:
a) a first IISRE sequence;
b) a second IISRE sequence;
c) an N-mer sequence;
d) a third IISRE sequence; and
e) an at least fourth IISRE sequence.

5. The Addamer of any one of the preceding claims, wherein the Addamer further comprises a multiple cloning site (MCS) sequence, wherein the MCS sequence comprises one or more restriction endonuclease sequences.

6. The Addamer of any one of the preceding claims, wherein at least one of the IISRE sequences are selected from a MlyI sequence, a NgoAVII sequence, SspD5I sequence, an AlwI sequence, a BccI sequence, a BcefI sequence, a PleI sequence, a BceAI sequence, a BceSIV sequence, a BscAI sequence, a BspD6I sequence, a FauI sequence, an EarI sequence, a BspQI sequence, a BfuAI sequence, a PaqCI sequence, an Esp3I sequence, a BbsI sequence, a BbvI sequence, a BtgZI sequence, a FokI sequence, a BsmFI sequence, a BsaI sequence, a BcoDI sequence and a HgaI sequence.

7. The Addamer of any one of the preceding claims, wherein at least one hairpin structure comprises an aptamer sequence.

8. The Addamer of claim 7, wherein the aptamer sequence is selected from a pL1 aptamer sequence, a Thrombin 29-mer aptamer sequence, an S2.2 aptamer sequence, an ART1172 aptamer sequence, an R12.45 aptamer sequence, a Rb008 aptamer sequence and a 38NT SELEX aptamer sequence.

9. A composition comprising the Addamer of any one of the preceding claims immobilized to a solid support.

10. The composition of claim 9, wherein the solid support is a bead.

11. The composition of claim 10, wherein the bead comprises polyacrylamide, polystyrene, agarose or any combination thereof.

12. The composition of claim 9, wherein the solid support is the surface of a well or chamber.

13. The composition of claim 12, wherein the well or chamber is part of a multi-well plate.

14. The composition of any one of claims 9-13, wherein
the Addamer comprises a hairpin structure comprising at least one aptamer sequence,
the solid surface comprises at least one ligand that binds to the aptamer sequence, and
the Addamer is immobilized to the solid surface via binding of the at least one aptamer sequence to the at least one ligand.

15. The composition of any one of claims 9-13, wherein
the Addamer comprises at least one 5′ overhang or 3′ overhang,
the solid surface comprises at least one single-stranded or partially double-stranded nucleic acid molecule with a single-stranded portion that is complementary to the at least one 5′ overhang or 3′ overhang, and
the Addamer is immobilized to the solid surface by hybridizing the at least one 5′ overhang or 3′ overhang to the at least one single-stranded or partially double-stranded nucleic acid on the solid surface.

16. The composition of any one of claims 9-13, wherein
the Addamer comprises at least one 5′ overhang or 3′ overhang,
the solid surface comprises at least one single-stranded or partially double-stranded nucleic acid molecule with a single-stranded portion that is complementary to the at least one 5′ overhang or 3′ overhang, and
the Addamer is immobilized to the solid surface by hybridizing the at least one 5′ overhang or 3′ overhang to the at least one single-stranded or partially double-stranded nucleic acid on the solid surface and ligating the Addamer and the at least one single-stranded or partially double-stranded nucleic acid.

17. A method of producing the Addamer of any one of claims 1-8, the method comprising:
a) chemically synthesizing a first single-stranded nucleic acid molecule and a second single-stranded nucleic acid molecule, wherein the sequences of the first single-stranded nucleic acid molecule and second single-stranded nucleic acid molecule comprise portions of the Addamer that is to be produced,
wherein the first single stranded nucleic acid molecule comprises a first region that is complementary to a second region on the second single-stranded nucleic acid molecule and the second region that is self-complementary, and
the second single-stranded nucleic acid molecule comprises a first region that is self-complementary and a second region that is complementary to a first region on the first single-stranded nucleic acid molecule;
b) hybridizing the first single-stranded nucleic acid and the second single-stranded nucleic acid to produce a partially double-stranded nucleic acid molecule; and
c) contacting the partially double-stranded nucleic acid molecule with a ligase enzyme to form a double-stranded Addamer structure capped at both ends by hairpins.

18. The method of claim 17, further comprising treating the products of step (c) with an exonuclease, thereby purifying properly ligated Addamers.

19. The method of claim 17 or claim 18, further comprising, after step (b) and before step (c), contacting the partially double-stranded nucleic acid molecule with a MutS enzyme.

20. A method of producing the Addamer of any one of claims 1-8, the method comprising:
a) cloning the Addamer sequence into a phagemid such that the Addamer sequence is flanked on both sides by one or more DNAzymes that can be selectively activated;
b) converting the phagemid into packaged bacteriophage using a helper phage, wherein the packaged bacteriophage produces single-stranded DNA comprising the Addamer sequence flanked on both sides by one or more DNAzymes;
c) purifying the single-stranded DNA produced by the packaged bacteriophage;
d) allowing the purified single-stranded DNA to fold to produce the one or more DNAzyme structure and a majority portion of the double-stranded Addamer sequence;
e) activating the one or more DNAzymes, thereby excising the Addamer sequence from the single-stranded DNA produced by the packaged bacteriophage; and
f) contacting the excised Addamer with a ligase enzyme.

21. The method of claim 20, further comprising treating the products of step (f) with exonuclease, thereby purifying properly ligated Addamers.

22. A method of producing the Addamer of any one of claims 1-8, the method comprising:
a) cloning the Addamer sequence into a plasmid;
b) propagating the plasmid in a suitable host organism;
c) purifying the plasmid from the host organism;
d) treating the purified plasmid with one or more of a nickase enzyme and a restriction endonuclease enzyme to excise the Addamer sequences from the plasmid; and
e) contacting the excised Addamer sequences with ligase to produce double-stranded Addamer structures capped at both ends by a hairpin structure.

23. A method of synthesizing a nucleic acid molecule comprising a target nucleic acid sequence, the method comprising:
a) providing a first Addamer of any one of claims 1-8 that is immobilized to a solid support, wherein the first Addamer comprises a first IISRE sequence, followed by a first N-mer sequence, followed by a second IISRE sequence, followed by a hairpin structure;
b) providing a second Addamer of any one of claims 1-8 that is immobilized to a solid support, wherein the second Addamer comprising a third IISRE sequence, followed by a second N-mer sequence, followed by a fourth IISRE sequence, followed by a hairpin structure;
c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end;
d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure;
e) ligating the first Cleaved Product and the Second Cleaved Product using a ligase enzyme to produce a first Ligation Product;
f) treating the products of step (e) with an exonuclease, thereby removing non-ligated first Cleaved Product and/or second Cleaved Product; and
g) repeating steps any combination of steps (a)-(f) using the products of step (f) and/or one or more additional Addamers of any one of claims 1-8 until the nucleic acid molecule comprising the target nucleic acid sequence has been synthesized.

24. The method of claim 23, wherein the ligase enzyme is human DNA ligase III (hLig3).

25. The method of claim 23, wherein the ligase enzyme is T4 DNA ligase.

26. The method of any one of claims 23-25, wherein the target nucleic acid sequence is at least about 100, or at least about 500, or at least about 1000, or at least about 2000, or at least about 3000, or at least about 4000, at least about 5000 nucleotides in length.

27. The method of any one of claims 23-26, wherein the synthesized nucleic acid molecule comprising the target nucleic acid sequence has a purity of at least 80%.

28. The method of any one of claims 23-27, wherein the synthesized nucleic acid molecule comprising the target nucleic acid sequence has a purity of at least 90%.


SEQUENCE LISTING (75 sequences)

SEQ ID NO: 1 - DNA, 61 bp, Artificial Sequence
  Sythetic Sequence
  misc_feature (41)..(43): n is a, c, g, or t
ttccttttgaattcccgggatccaagcttaaagaagacccnnncatcggactcaaaaagtt

SEQ ID NO: 2 - DNA, 58 bp, Artificial Sequence
  Sythetic Sequence
  misc_feature (39)..(41): n is a, c, g, or t
tggaaaacttaagggccctaggttcgaattcttctgggnnngtagcctgagtttttct

SEQ ID NO: 3 - DNA, 69 bp, Artificial Sequence
  Sythetic Sequence
  misc_feature (49)..(51): n is a, c, g, or t
ttccttttgaattcccgggatccaagcttaaagcgatgtctagatcggnnncatcggactcaaaaagtt

SEQ ID NO: 4 - DNA, 67 bp, Artificial Sequence
  Sythetic Sequence
  misc_feature (48)..(50): n is a, c, g, or t
tggaaaacttaagggccctaggttcgaatttcgctacagatctagccnnngtagcctgagtttttct

SEQ ID NO: 5 - DNA, 63 bp, Artificial Sequence
  Sythetic Sequence
  misc_feature (43)..(45): n is a, c, g, or t
ttccttttgaattcccgggatccaagcttaaaacctgctctgnnncatcggactcaaaaagtt

SEQ ID NO: 6 - DNA, 61 bp, Artificial Sequence
  Sythetic Sequence
  misc_feature (42)..(44): n is a, c, g, or t
tggaaaacttaagggccctaggttcgaattttggacgagacnnngtagcctgagtttttct

SEQ ID NO: 7 - DNA, 63 bp, Artificial Sequence
  Sythetic Sequence
  misc_feature (43)..(45): n is a, c, g, or t
ttcctttgaattcccgggatccaagcttaaacacctgctctgnnncatcggactcaaaaagtt

SEQ ID NO: 8 - DNA, 62 bp, Artificial Sequence
  Sythetic Sequence
  misc_feature (43)..(45): n is a, c, g, or t
tggaaaacttaagggccctaggttcgaatttgtggacgagacnnngtagcctgagtttttct

SEQ ID NO: 9 - DNA, 67 bp, Artificial Sequence
  Sythetic Sequence
  misc_feature (47)..(49): n is a, c, g, or t
ttccttttgaattcccgggatccaagcttaaaggatgtctagatcgnnncatcggactcaaaaagtt

SEQ ID NO: 10 - DNA, 65 bp, Artificial Sequence
  Sythetic Sequence
  misc_feature (46)..(48): n is a, c, g, or t
tggaaaacttaagggccctaggttcgaatttcctacagatctagcnnngtagcctgagtttttct

SEQ ID NO: 11 - DNA, 60 bp, Artificial Sequence
  Sythetic Sequence
  misc_feature (40)..(42): n is a, c, g, or t
ttccttttgaattcccgggatccaagcttaaactcttcgnnncatcggactcaaaaagtt

SEQ ID NO: 12 - DNA, 58 bp, Artificial Sequence
  Sythetic Sequence
  misc_feature (39)..(41): n is a, c, g, or t
tggaaaacttaagggccctaggttcgaatttgagaagcnnngtagcctgagtttttct

SEQ ID NO: 13 - DNA, 16 bp, Artificial Sequence
  Sythetic Sequence
aaagcagcgaagactg

SEQ ID NO: 14 - DNA, 20 bp, Artificial Sequence
  Sythetic Sequence
tttcgtcgcttctgacacgt

SEQ ID NO: 15 - DNA, 10 bp, Artificial Sequence
  Sythetic Sequence
gggacgagtc

SEQ ID NO: 16 - DNA, 11 bp, Artificial Sequence
  Sythetic Sequence
gcgatggagtc

SEQ ID NO: 17 - DNA, 11 bp, Artificial Sequence
  Sythetic Sequence
ggtgagaagac

SEQ ID NO: 18 - DNA, 11 bp, Artificial Sequence
  Sythetic Sequence
gccgccgtctc

SEQ ID NO: 19 - DNA, 11 bp, Artificial Sequence
  Sythetic Sequence
gccgcggtctc

SEQ ID NO: 20 - DNA, 11 bp, Artificial Sequence
  Sythetic Sequence
  misc_feature (6)..(6): n is a, c, g, or t
gccgcngtctc

SEQ ID NO: 21 - DNA, 10 bp, Artificial Sequence
  Sythetic Sequence
acggcgccgc

SEQ ID NO: 22 - DNA, 12 bp, Artificial Sequence
  Sythetic Sequence
  misc_feature (6)..(7): n is a, c, g, or t
acggcnngagtc

SEQ ID NO: 23 - DNA, 12 bp, Artificial Sequence
  Sythetic Sequence
  misc_feature (6)..(6): n is a, c, g, or t
ggtganctcttc

SEQ ID NO: 24 - DNA, 12 bp, Artificial Sequence
  Sythetic Sequence
ggtgagctcttc

SEQ ID NO: 25 - DNA, 13 bp, Artificial Sequence
  Sythetic Sequence
  misc_feature (6)..(7): n is a, c, g, or t
gggacnngaagac

SEQ ID NO: 26 - DNA, 15 bp, Artificial Sequence
  Sythetic Sequence
  misc_feature (7)..(9): n is a, c, g, or t
gcgatgnnncgtctc

SEQ ID NO: 27 - DNA, 13 bp, Artificial Sequence
  Sythetic Sequence
  misc_feature (6)..(7): n is a, c, g, or t
ggatgnnggtctc

SEQ ID NO: 28 - DNA, 12 bp, Artificial Sequence
  Sythetic Sequence
  misc_feature (6)..(7): n is a, c, g, or t
gcagcnngtctc

SEQ ID NO: 29 - DNA, 68 bp, Artificial Sequence
  Sythetic Sequence
  misc_feature (35)..(37): n is a, c, g, or t
ccgggatccaagcttaaagcgatgtctagatcggnnncatcggactcaaaaagtttctttttgagtcc

SEQ ID NO: 30 - DNA, 68 bp, Artificial Sequence
  Sythetic Sequence
  misc_feature (62)..(64): n is a, c, g, or t
cttaagttttcctttggaaaacttaagggccctaggttcgaatttcgctacagatctagccnnngtag

SEQ ID NO: 31 - DNA, 68 bp, Artificial Sequence
  Sythetic Sequence
  misc_feature (48)..(50): n is a, c, g, or t
ttccttttgaattcccgggatccaagcttaaagcgatgtctagatcgnnncatcggactcaaaaagtt

SEQ ID NO: 32 - DNA, 66 bp, Artificial Sequence
  Sythetic Sequence
  misc_feature (47)..(49): n is a, c, g, or t
tggaaaacttaagggccctaggttcgaatttcgctacagatctagcnnngtagcctgagtttttct

SEQ ID NO: 33 - DNA, 27 bp, Artificial Sequence
  Sythetic Sequence
gacatgagggtgcccgctcagctcctg

SEQ ID NO: 34 - DNA, 11 bp, Artificial Sequence
  Sythetic Sequence
gacatgagggt

SEQ ID NO: 35 - DNA, 11 bp, Artificial Sequence
  Sythetic Sequence
ctgtactccca

SEQ ID NO: 36 - DNA, 12 bp, Artificial Sequence
  Sythetic Sequence
gggtgcccgctc

SEQ ID NO: 37 - DNA, 12 bp, Artificial Sequence
  Sythetic Sequence
cccacgggcgag

SEQ ID NO: 38 - DNA, 12 bp, Artificial Sequence
  Sythetic Sequence
gctcagctcctg

SEQ ID NO: 39 - DNA, 12 bp, Artificial Sequence
  Sythetic Sequence
cgagtcgaggac

SEQ ID NO: 40 - DNA, 12 bp, Artificial Sequence
  Sythetic Sequence
gggtgcccgctc

SEQ ID NO: 41 - DNA, 12 bp, Artificial Sequence
  Sythetic Sequence
cccacgggcgag

SEQ ID NO: 42 - DNA, 12 bp, Artificial Sequence
  Sythetic Sequence
cccacgggcgag

SEQ ID NO: 43 - DNA, 12 bp, Artificial Sequence
  Sythetic Sequence
gctcagctcctg

SEQ ID NO: 44 - DNA, 12 bp, Artificial Sequence
  Sythetic Sequence
cgagtcgaggac

SEQ ID NO: 45 - DNA, 12 bp, Artificial Sequence
  Sythetic Sequence
gctcagctcctg

SEQ ID NO: 46 - DNA, 20 bp, Artificial Sequence
  Sythetic Sequence
gggtgcccgctcagctcctg

SEQ ID NO: 47 - DNA, 20 bp, Artificial Sequence
  Sythetic Sequence
cccacgggcgagtcgaggac

SEQ ID NO: 48 - DNA, 11 bp, Artificial Sequence
  Sythetic Sequence
gacatgagggt

SEQ ID NO: 49 - DNA, 11 bp, Artificial Sequence
  Sythetic Sequence
ctgtactccca

SEQ ID NO: 50 - DNA, 11 bp, Artificial Sequence
  Sythetic Sequence
ctgtactccca

SEQ ID NO: 51 - DNA, 20 bp, Artificial Sequence
  Sythetic Sequence
gggtgcccgctcagctcctg

SEQ ID NO: 52 - DNA, 20 bp, Artificial Sequence
  Sythetic Sequence
cccacgggcgagtcgaggac

SEQ ID NO: 53 - DNA, 20 bp, Artificial Sequence
  Sythetic Sequence
gggtgcccgctcagctcctg

SEQ ID NO: 54 - DNA, 16 bp, Artificial Sequence
  Sythetic Sequence
cgggcgagtcgaggac

SEQ ID NO: 55 - DNA, 27 bp, Artificial Sequence
  Sythetic Sequence
gacatgagggtgcccgctcagctcctg

SEQ ID NO: 56 - DNA, 27 bp, Artificial Sequence
  Sythetic Sequence
ctgtactcccacgggcgagtcgaggac

SEQ ID NO: 57 - DNA, 23 bp, Artificial Sequence
  Sythetic Sequence
gacatgagggtgcccgctcagct

SEQ ID NO: 58 - DNA, 23 bp, Artificial Sequence
  Sythetic Sequence
actcccacgggcgagtcgaggac

SEQ ID NO: 59 - DNA, 25 bp, Artificial Sequence
  Sythetic Sequence
ccaaatgcgtgacagtcttcggcga

SEQ ID NO: 60 - DNA, 25 bp, Artificial Sequence
  Sythetic Sequence
ggtttacgcactgtcagaagccgaa

SEQ ID NO: 61 - DNA, 12 bp, Artificial Sequence
  Sythetic Sequence
ggtttacgcact

SEQ ID NO: 62 - DNA, 33 bp, Artificial Sequence
  Sythetic Sequence
ccaaatgcgtgaatgctgtccagtcttcggcga

SEQ ID NO: 63 - DNA, 33 bp, Artificial Sequence
  Sythetic Sequence
ggtttacgcacttacgacaggtcagaagccgaa

SEQ ID NO: 64 - DNA, 16 bp, Artificial Sequence
  Sythetic Sequence
ccaaatgcgtgaatgc

SEQ ID NO: 65 - DNA, 20 bp, Artificial Sequence
  Sythetic Sequence
ggtttacgcacttacgacag

SEQ ID NO: 66 - DNA, 32 bp, Artificial Sequence
  Sythetic Sequence
  misc_feature (25)..(32): n is a, c, g, or t
ccaaatgcgtgaatgctgtcgactnnnnnnnn

SEQ ID NO: 67 - DNA, 20 bp, Artificial Sequence
  Sythetic Sequence
ggtttacgcacttacgacag

SEQ ID NO: 68 - DNA, 32 bp, Artificial Sequence
  Sythetic Sequence
tcgattggattgtgccggaagtgctggctcga

SEQ ID NO: 69 - DNA, 29 bp, Artificial Sequence
  Sythetic Sequence
agtccgtggtagggcaggttggggtgact

SEQ ID NO: 70 - DNA, 23 bp, Artificial Sequence
  Sythetic Sequence
cagttgatcctttggataccctg

SEQ ID NO: 71 - DNA, 42 bp, Artificial Sequence
  Sythetic Sequence
ggcgtgcagtgccttcggccgtgcggtgcctccgtcacgcct

SEQ ID NO: 72 - DNA, 43 bp, Artificial Sequence
  Sythetic Sequence
accgtctgagcgattcgtactttattcgggaggtatcagcggg

SEQ ID NO: 73 - DNA, 34 bp, Artificial Sequence
  Sythetic Sequence
cctggacggaaccagaatacttttggtctccagg

SEQ ID NO: 74 - DNA, 43 bp, Artificial Sequence
  Sythetic Sequence
aaatacccccccttcggtgcaaagcaccgaagggggggtattt

SEQ ID NO: 75 - DNA, 11 bp, Artificial Sequence
  Sythetic Sequence
gacatgagggt